(19) United States
(12) Patent Application Publication
(10) Pub. No.: US 2026/0144760 A1
(43) Pub. Date: May 28, 2026

(54) METHOD FOR TREATING LOCAL AND DISTANT TUMORS

(71) Applicant: IntraAb, Inc., Richmond, CA (US)

(72) Inventors: Lijun Wu, Berkeley, CA (US); Vita Golubovskaya, Pinole, CA (US)

(21) Appl. No.: 19/459,832

(22) Filed: Jan. 26, 2026

Related U.S. Application Data
(63) Continuation of application No. PCT/US2024/039565, filed on Jul. 25, 2024.
(60) Provisional application No. 63/515,769, filed on Jul. 26, 2023.
(60) Provisional application No. 63/653,460, filed on May 30, 2024.

(51) Int. Cl. A61K 9/51; A61K 38/19; A61K 38/20; A61K 39/395; A61P 35/00

(52) U.S. Cl. CPC A61K 9/5123; A61K 38/193; A61K 38/208; A61K 39/39558; A61P 35/00

(57) ABSTRACT

The present invention is directed to a method for treating cancer by delivering lipid-nanoparticles (LNPs) encapsulating immunostimulant mRNA and bispecific antibody mRNA to cancer cells or intratumorally. The method comprises the step of administering mRNA-encapsulated LNPs to a tumor lesion of a subject having cancer. The immunostimulant mRNA-LNP activates immune cells around tumor, which work together with mRNA-LNP encoding bispecific antibody and PBMC to effectively target local and distant metastatic tumors. Preferred immunostimulants are GM-CSF and IL-12, and its combination.

### CROSS-REFERENCE TO RELATED APPLICATION(S)

This application is a continuation of PCT/US2024/039565, filed Jul. 25, 2024; which claims priority to US Provisional Application Nos. 63/515,769, filed Jul. 26, 2023; and 63/653,460, filed May 30, 2024. The contents of the above-identified applications are incorporated herein by reference in their entirety.

### REFERENCE TO SEQUENCE LISTING, TABLE OR COMPUTER PROGRAM

This application contains an ST.26 compliant Sequence Listing, which was submitted in xml format via Patent Center and is hereby incorporated by reference in its entirety. The .xml copy, created on Jul. 25, 2024, is named 1199958057WO01SequenceListing.xml and is 66800 bytes in size.

### TECHNICAL FIELD

The present invention relates to generating mRNA-LNP (lipid nanoparticle) delivery of immunostimulants, an antibody against tumor antigen, and a T cell engager to cancer or tumor cells to activate immune cells (T cells, NK cells, dendritic cells, macrophages) and to attack local and distant tumor cells. mRNA-LNP of immunostimulants delivered to tumors results in expression of tethered proteins (expressed on cell surface) or secreted proteins and leads to local and distant tumor regression.

### BACKGROUND OF THE INVENTION

Immunotherapy is emerging as a highly promising approach for the treatment of cancer. T cells or T lymphocytes, the armed forces of our immune system, constantly look for foreign antigens and discriminate abnormal (cancer or infected cells) from normal cells. There are several immunotherapy approaches such as: 1. monoclonal antibodies; 2. immune checkpoint inhibitors; 3. cancer vaccines; 4. CAR-T, CAR-NK cells immunotherapies.

Tumor vaccines can effectively deliver tumor-specific antigens (TSAs) to antigen-presenting cells (APCs) and activate tumor-specific T cells. They can establish long-lasting antitumor memory and induce local tumor regression and eradication of distant metastatic lesions. The failure of tumor vaccines can be attributed to insufficient tumor immunogenicity, which prevents the generation of sufficient robust T cells that are required to induce long-term immunity. One of the approaches is to improve the efficacy of tumor-derived is co-deliver tumor antigens with natural, synthetic, or genetically engineered immune-stimulatory molecules.

### BRIEF DESCRIPTION OF THE DRAWINGS

FIG. 1 shows a scheme of DNA vector template (A) used for in vitro transcription of protein or bispecific antibody RNA (B). 5′UTR, 5′ untranslated region; 3′UTR, 3′untranslated region; poly A tail for increased stability.

FIG. 2 shows a structure of EPCAM scFv-CD3 ScFv-human Fc. The human Fc may contain mutations L234A, L235A and P239G to decrease Fc-dependent ADCC effects.

FIG. 3 shows GM-CSF protein expression detected by ELISA after transfection of mRNA-LNP into HEK-293 cells. Supernatant was collected from transfected cells and used for protein detection with GM-CSF ELISA kit.

FIG. 4 shows secretion of IL-12 from transfected with IL-12 mRNA-LNP 293 cells, detected by ELISA.

FIG. 5 shows increased expansion of NK cells after transfecting IL-12 mRNA-LNP into cells. NK cells after transfection of IL12 mRNA-LNP resulting in secretion of IL-12 into medium expanded significantly more than NK cells with no cytokines in the medium when NK cells died.

FIG. 6 shows increased IFN-gamma secretion by immunostimulant mRNA-LNP transfected to OVCAR-5 cells in killing assay with EpCAM-CD3 antibody and PBMC cells.

FIGS. 7A-7B show efficacy of EpCAM-CD3-mRNA-LNP at local and distant tumor sites. EpCAM-CD3 injected intratumorally into the left side had blocked tumor growth only at the left side (7A), but not at the untreated with RNA-LNP right side (7B).

FIGS. 8A-8B show four-immunostimulant mix mRNA-LNP decreased tumor growth in mice treated with EpCAM-CD3-hFc mRNA-LNP and T cells at left side (local delivery) (8A); and also at distant untreated right side (8B). *p<0.05: EpCAM-CD3 hFc mRNA-LNP+Mix 1 or Mix 2 vs. EGFP mRNA-LNP both right and left sides by Student's t-test.

FIGS. 9A-9B show immunostimulant-Mix 1A (IL-12 and GM-CSF) mRNA-LNP decreased tumor growth in treated with EpCAM-CD3-hFc mRNA-LNP and PBMC cells mice at left side (local delivery) (9A), and also at distant untreated right side (9B). FIGS. 9C-9D show immunostimulant Mix 1B (CXCL-9+PD-L1) did not decrease tumor growth at local-treated left side (9C), and did not decrease at distant untreated right side (9D). *p<0.05 EpCAM-CD3 hFc mRNA-LNP+Mix 1A versus GFP mRNA-LNP both right and left sides by Student's t-test.

FIGS. 10A-10B show that IL-12 mRNA-LNP and GM-CSF mRNA-LNP with EpCAM-CD3 mRNA-LNP and PBMC significantly inhibited tumor growth, in both local site (10A) and distant (10B) side. In 10B, *p<0.05: EpCAM-CD3 mRNA-LNP+IL-12 mRNA-LNP +GM-CSF mRNA-LNP vs. EpCAM-CD3 mRNA-LNP+IL-12 mRNA-LNP, and vs. EpCAM-CD3 mRNA-LNP+GM-CSF mRNA-LNP at days 21-28 at the distant right side, Student's t-test. Also, **p<0.05: EpCAM-CD3 mRNA-LNP+IL-12 mRNA-LNP+GM-CSF mRNA-LNP vs. EpCAM-CD3 mRNA-LNP+IL-12 mRNA-LNP, and vs. EpCAM-CD3 mRNA-LNP+GM-CSF mRNA-LNP at day 38 at the distant right side.

FIG. 11 shows that intratumoral delivery of (i) Mesothelin-CD3hFc mRNA, and (ii) Her-2-CD3hFc mRNA-LNP, with IL-12 mRNA-LNP and GM-CSF mRNA-LNP significantly blocked more tumor growth in A1847 model than each bispecific mRNA-LNP alone. *p<0.05 bispecific antibody+IL-12+GM-CSF versus bispecific antibody alone, Student's t-test.

### DETAILED DESCRIPTION OF THE INVENTION

### Definitions

As used herein, “activated T cells” are T cells activated for proliferation and cell killing.

As used herein, a “domain” means one region in a polypeptide which is folded into a particular structure independently of other regions.

As used herein, “immunostimulants”, also known as immunostimulators, are substances (drugs, molecules, antibodies and proteins) that stimulate the immune system by inducing activation or increasing activity of any of its components.

As used herein, a “tumor antigen” means a biological molecule having antigenicity, expression of which causes cancer.

In general, mRNA is transient and short-lived when delivered in vivo. The present invention relates to a method for producing antibodies and immunostimulant molecules by delivering lipid nanoparticle-encapsulated mRNA to cancer/tumor cells. By encapsulating mRNA in lipid nanoparticles, the stability of mRNA is improved.

The present invention is directed to a method for treating cancer. In the first aspect, the method comprises the steps of: (a) obtaining first lipid nanoparticles (LNPs) encapsulating a first mRNA that encodes a bispecific antigen-binding molecule comprising a first ScFv and CD3e ScFv, wherein said first scFv is against EPCAM, Her-2, Mesothelin, EGFR, PLAP, CD147, 4-1BB, c-Met, CD19/CD37, PSMA, CD47, CD19, BCMA, or Claudin 18.2; (b) obtaining one or more second LNPs each encapsulating a second mRNA that encodes a check-point inhibitor, a cytokine, a chemokine, or an immunomodulator; and (c) administering the first mRNA-encapsulated LNPs and the second mRNA-encapsulated LNPs to a tumor lesion of a subject having cancer. In this method, the first mRNA-encapsulated LNPs and the second mRNA-encapsulated LNPs are administered to the subject simultaneously or sequentially.

In the second aspect, the method comprises the steps of: (a) obtaining lipid nanoparticles (LNPs) encapsulating (i) a first mRNA that encodes a bispecific antigen-binding molecule comprising a first ScFv and CD3e ScFv, wherein said first scFv is against EPCAM, Her-2, Mesothelin, PSMA, CD47, CD19, BCMA, Claudin 18.2, and (ii) a second mRNA that encodes one or more check-point inhibitor, cytokine, chemokine, and/or immunomodulator; and (b) administering the mRNA-encapsulated LNPs and the second mRNA-encapsulated LNPs to a tumor lesion of a subject having cancer. In this method, the first mRNA and the second mRNA—are co-expressed in the same LNPs.

Check-point inhibitors useful for the present invention include antibody or ligand to PD-L1, PD-1, TIGIT, LAG3, TIM3, and CTLA4.

Cytokines useful for the present invention include, but not limited to, IL-12, GM-CSF, IL-2, IL-15, and IFN-gamma.

Chemokines useful for the present invention include, but not limited to CXCL-9 and CCL-2.

Immunomodulator useful for the present invention include, but not limited to, OX-40, OX-40L, CD70, CD80, 4-1BB, CD40, and TLRs.

Preferred immunostimulants are IL12, GM-CSF, and the combination thereof.

In one preferred method, the method comprises: (i) obtaining first lipid nanoparticles (LNPs) encapsulating a first mRNA that encodes a bispecific antigen-binding molecule comprising a first ScFv and CD3e ScFv, wherein said first scFv is against EPCAM, Her-2, Mesothelin, EGFR, PLAP, CD147, 4-1BB, c-Met, CD19/CD37, PSMA, CD47, CD19, BCMA, or Claudin 18.2; (ii) obtaining second LNPs encapsulating a second mRNA that encodes GM-CSF; (iii) obtaining third LNPs encapsulating a third mRNA that encodes IL-12; and (iv) administering the first, the second, and the third mRNA-encapsulated LNPs s to a tumor lesion of a subject having cancer.

In another preferred method, the method comprises: (i) obtaining lipid nanoparticles (LNPs) encapsulating (a) a first mRNA that encodes a bispecific antigen-binding molecule comprising a first ScFv and CD3e ScFv, wherein said first scFv is against EPCAM, Her-2, Mesothelin, EGFR, PLAP, CD147, 4-1BB, c-Met, CD19/CD37, PSMA, CD47, CD19, BCMA, Claudin 18.2, (b) a second mRNA that encodes Gm-CSF, and (c) a third mRNA that encodes IL-12; and (ii) administering the mRNA-encapsulated LNPs to a tumor lesion of a subject having cancer.

The present method intratumorally delivers mRNA-immunostimulants to stimulate immune cells together with intratumoral delivery of mRNA-LNP encoding bispecific antibody to kill tumor, which results in effective vaccine to kill tumor.

The present methods activate T cells and/or NK cells in the tumor microenvironment to treat tumor at the administered local site and tumor at a distant site.

In one embodiment, the present method treats colorectal, ovarian, pancreatic, breast, or lung cancer.

In one embodiment, the present method treats metastatic solid cancer.

In one embodiment, the present method provides an abscopal effect in the treatment of metastatic cancer, in which shrinkage of distant tumors occurs concurrently with shrinkage of tumors within a localized treatment.

In one embodiment, the lipid nanoparticles comprise 8-[(2-hydroxyethyl)[6-oxo-6-(undecyloxy)hexyl]amino]-octanoic acid, 1-octylnonyl ester (SM-102), distearoylphosphatidylcholine (DSPC), Cholesterol, and 1,2-dimyristoyl-rac-glycero-3-methoxypolyethylene glycol-2000 (DMG-PEG2000).

Insertion of mRNA into LNP nanoparticles provides protection of mRNA from degradation and increases the stability mRNA; mRNA is then released from LNPs into cells in vivo to generate protein. mRNA-lipid nanoparticle preparation is described in Schoenmaker (International J. Pharmaceutics, 601: 120856, 2021), the article is incorporated herein by reference in its entirety, in particular regarding the LNPs.

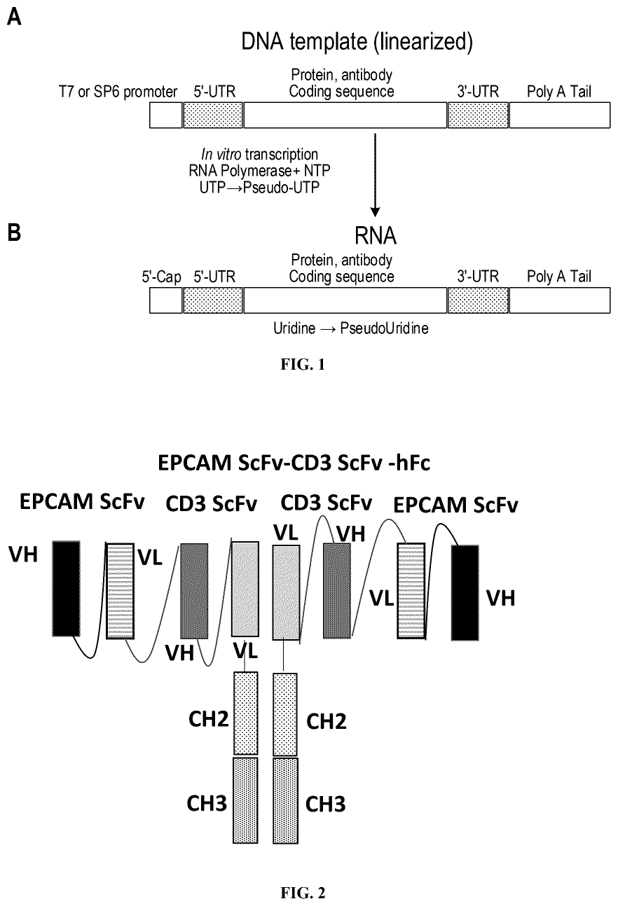

FIG. 1 shows linearized DNA template to be used for in vitro transcription with RNA polymerase and nucleotide triphosphate to generate protein or antibody mRNA. The DNA template contains T7AG promoter, then 5′UTR (untranslated region), the coding region of protein or antibody, then 3′UTR, and then >100 poly A tail for RNA stability. The generated mRNA contains 5′ cap ([m7G(5′)ppp(5′)G], cap1 for increased stability. The mRNA is embedded to lipid nanoparticles (LNPs) and can be either transfected to cancer/tumor cells to stimulate immune tumor microenvironment in vivo.

In the DNA sequence, the promoter is T7AG promoter. Poly A tail sequence is from 20-170 nucleotides. Poly A tail sequence can comprise one or more linkers in between the poly A segments. If poly A tail is longer than 60 nucleotides, then it typically contains a linker which includes non-adenosine nucleotides. A linker is 5-30 or 5-25 nucleotides, e.g., 10 nucleotides or 20 nucleotides. In one example, poly A tail is 150-160 nucleotides in length, consisting of two linker sequences.

DNA expression is finely regulated at the post-transcriptional level. Untranslated regions are not translated into amino acids; however, UTRs of mRNAs may control their translation, degradation and localization include stem-loop structures, upstream initiation codons and open reading frames, internal ribosome entry sites and various cis-acting elements that are bound by RNA-binding proteins. UTRs are important in the post-transcriptional regulation of DNA expression, including modulation of the transport of mRNAs out of the nucleus and of translation efficiency, subcellular localization, and stability.

5′-UTR typically has 10-1000 nucleotides, or 20-500 nucleotides, or 30-200 nucleotides, or 30-100 nucleotides. For example, 5′-UTR is 50 nucleotides. 3′-UTR typically has 10-3000 nucleotides, for example, 50-500 nucleotides, or 100-300 nucleotides. Preferred 5′-UTRs and 3′-UTRs are UTRs of 3-globin, or UTRs of Pfizer COVID vaccine.

β-Globin gene is shown in:
https://www.ncbi.nlm.nih.gov/nucleotide/V00497.1?report=genbank&log$=nuclalign&blast_ran k=5&RID=TDDZ1K98016

In Pfizer COVID vaccine, the 5′-untranslated region is derived from human alpha-globin RNA with an optimized Kozak sequence. The 3′ untranslated region comprises two sequence elements derived from the amino-terminal enhancer of split (AES) mRNA and the mitochondrial encoded 12S ribosomal RNA to confer RNA stability and high total protein expression.

Any suitable vector, such as Vector pSP64 Poly(A) (Promega) or pGEM3Z-Vector (Promega) can be used as a cloning vector for the DNA sequence described above.

For example, to engineer the pEM3Z-β-globin UTR-UTR-poly A tail, the 3′-UTR of the β-globin molecule flanked by restriction enzyme site can be amplified from human bone marrow. For example, a single (pEM3Z-1β-globin-UTR-A[120]) or 2 serial fragments (pEM3Z-20-globin-UTR-A[120]) can be inserted in front of the poly(A) tail.

In one embodiment, bispecific EpcAM-CD3-human Fc antibody RNAs are used with LNP to deliver to mammalian 293 cells or OVCAR-5 cells and secreted antibody with T cells used to kill cancer cells.

EPCAM is an epithelial cell adhesion molecule that is encoded by EPCAM gene. EpCAM is a cell surface glycoprotein of approximately 40 kDa which is highly expressed in epithelial cancers and has lower expression in normal epithelial tissues. There are several names of EPCAM such as TROP1, CD326, HEA125, EGP40, KSA, ESA. EPCAM regulates cell-cell contact adhesions and tissue plasticity, and controls cell proliferation and differentiation.

As used herein, “CD3 epsilon (CD3e)” is a polypeptide encoded by the CD3E gene which resides on chromosome 11 in human. CD3-epsilon polypeptide, which together with CD3-gamma, -delta and -zeta, and the T-cell receptor alpha/beta and gamma/delta heterodimers, forms the T cell receptor-CD3 complex. This complex plays an important role in coupling antigen recognition to several intracellular signal-transduction pathways. The CD3 epsilon polypeptide plays an essential role in T-cell development. CD3 epsilon, CD3e, and CD3 are used interchangeably in this application.

FIG. 2 illustrates an example of a bispecific antibody structure suitable for the present invention. FIG. 2 uses EPCAM as an example, but the same structure applies to other bispecific antibodies described in the present application.

FIG. 2 shows the structure of EPCAM-CD3 epsilon chain (CD3e) bispecific antigen-binding molecule, in which the bispecific antigen-binding molecule comprises a monovalent humanized anti-EPCAM ScFv and a monovalent CD3e ScFv fused to human Fc; the structure consists of one DNA constructs. The Fc has L234L235A and P329G mutations for decreasing Fc-dependent immune response. The structure has an EPCAM scFv and CD3e scFv which are connected by a linker and then human Fc fused to CD3 Scfv for increased stability. The bispecific antibody (BITE-human Fc format) comprises one binding moiety to EPCAM, and one binding moiety to CD3 epsilon. The antibody had a dimeric conformation.

In FIG. 2, the bispecific antigen-binding molecule comprises EPCAM VH, a first linker, EPCAM VL, a second linker, CD3 VH, a third linker, CD3 VL, and a human Fc domain, wherein the CH2 of the human Fc domain optionally comprises one or more amino acid substitutions selected from the group consisting of L234, L235, and P329 (EU numbering). The first, the second, and the third, and the fourth linkers may be the same or different.

In one embodiment, the linkers of the present bispecific antigen-binding molecules have the amino acid sequence of GGGGS (SEQ ID NO: 1), which is repeated n times, and n=1-5. In one preferred embodiment, n=3.

The DNAs of the bispecific antigen-binding molecule, and the DNAs of the check-point inhibitor, cytokine, chemokine, and/or immunomodulator are inserted into DNA template vectors with either T7AG promoter for RNA polymerase to generate mRNAs by in vitro transcription. The mRNAs are mixed with lipid components to produce lipid nanoparticles (LNPs) with mRNA encapsulated. Then LNP-encapsulated mRNAs are transfected into mammalian cells to translate mRNAs inside cells to produce the antibody protein and the check-point inhibitor, cytokine, chemokine, and/or immunomodulator, which work together to kill tumor cells locally or distantly.

In one embodiment, the DNAs of the bispecific antigen-binding molecule of EPCAM and CD3e, and the DNAs of IL-12 and/or GM-CSF are inserted into DNA template vectors for RNA polymerase to generate mRNAs by in vitro transcription. The mRNAs are mixed with lipid components to produce lipid nanoparticles (LNPs) with mRNA encapsulated. Then LNP-encapsulated mRNAs are transfected into mammalian cells to translate mRNAs inside cells to produce the antibody proteins, IL-12, and/or GM-CSF, which work together to kill tumor cells locally or distantly. IL-12 alone is able to decrease distant tumor growth. The combination of IL-12 and GM-CSF works better in decreasing tumor growth, locally or distantly.

The following examples further illustrate the present invention. These examples are intended merely to be illustrative of the present invention and are not to be construed as being limiting.

### EXAMPLES

### Example 1. Preparation of Linearized DNA Template for In Vitro Transcription

DNA was digested with appropriate restriction Bgl II (AGATCT) or Asc I (GGCGCGCC) enzyme which cut DNA right 3′ after poly A tail at 37° C. overnight following manufacturer's protocol. Then digested DNA was treated with 50-100 g/mL Proteinase K and 0.5% SDS for 30 minutes at 50° C. Then phenol/chloroform extraction and ethanol precipitation of DNA was performed. The DNA was used for in vitro RNA transcription reaction.

### Example 2. Preparation of RNA by In Vitro Transcription Reaction

For DNA templates with SP6 promoter, we used the below protocol. 2.1. The in vitro transcription reaction was done by below protocol:

When DNA template for generating RNA had T7AG promoter in front of protein or antibody coding sequence, the Standard RNA Synthesis Protocol using the HiScribe T7 mRNA Kit with CleanCap Reagent AG (NEB #E2080) was used and described below:
1. Set up the following reaction at room temperature in the following order:

| | 20 μl | Final conc. or |
|---|---|---|
| Components | reaction | amount |
| Nuclease-free water | X μl | |
| 10X T7 CleanCap Reagent AG | 2 μl | |
| Reaction Buffer | | |
| ATP (60 mM) | 2 μl | 6 mM final |
| UTP (50 mM) | 2 μl | 5 mM final |
| CTP (50 mM) | 2 μl | 5 mM final |
| GTP (50 mM) | 2 μl | 5 mM final |
| Cap Analog (40 mM) | 2 μl | 4 mM final |
| Template DNA | X μl | 1 μg |
| T7 RNA Polymerase Mix | | 2 μl |

2. Gently mix the reaction by pipetting up and down and microfuge briefly. Incubate at 37° C. for 2 hours.
3. Bring the reaction volume up to 50 μl with nuclease-free water. Add 2 μl of DNase I, mix well and incubate at 37° C. for 15 minutes
4. Proceed with mRNA purification

### 2.2. Cleaning In Vitro Transcribed RNA

For cleaning RNA, we used MEGAclear™ Kit (Thermofisher AM1908) following below protocol:
1. Bring the RNA sample to 100 μL with Elution Solution and mix.
2. Add 350 μL of Binding Solution Concentrate to the sample and mix.
3. Add 250 μL of 100% ethanol to the sample.
4. Apply the sample to the filter:
   a. Insert a Filter Cartridge into one of the Collection and Elution Tubes supplied.
   b. Pipet the RNA mixture onto the Filter Cartridge.
   c. Centrifuge for ˜15 sec to 1 min, or until the mixture has passed through the filter. Centrifuge at 10,000-15,000×g (typically 10,000-14,000 rpm).
   d. Discard the flow-through and reuse the Collection and Elution Tube for the washing steps.
5. Wash with 2×500 μL Wash Solution.
6. Elute RNA from the filter with 50 μL Elution Solution
   a. Pre-heat 110 μL of Elution Solution per sample to 95° C.
   b. Apply 50 μL of the pre-heated Elution Solution to the center of the Filter Cartridge, close the cap of the tube and centrifuge for 1 min at room temperature (RCF 10,000-15,000×g) to elute the RNA.
   c. To maximize RNA recovery, repeat this elution procedure with a second preheated 50 μL aliquot of Elution Solution. Collect the eluate into the same Collection/Elution Tube.

### 2.3. Assessing RNA Yield

The concentration of RNA is determined by diluting an aliquot of the preparation (usually a 1:50 to 1:100 dilution) in 1×TE (10 mM Tris-HCl pH 8, 1 mM EDTA) buffer, and reading the absorbance in a spectrophotometer at 260 nm. The concentration (μg/mL) of RNA is therefore calculated as follows: A260×dilution factor×40 μg/mL.

### Example 3. Preparation of LNPs

### Lipids:

SM-102: 8-[(2-hydroxyethyl)[6-oxo-6-(undecyloxy)hexyl]amino]-octanoic acid, 1-octylnonyl ester; CAS number: 2089251-47-6
DMG-PEG2000: 1,2-dimyristoyl-rac-glycero-3-methoxypolyethylene glycol-2000
DSPC: Distearoylphosphatidylcholine
Cholesterol

### SM-102 Formulation:

The SM-102 formulation was prepared using SM-102:DSPC:Cholesterol:DMG-PEG2000=(50:10:38.5:1.5 mol %). In other experiments, different ratios and different lipid components can be used to make LNPs.

### Example 4. RNA Encapsulation to LNPs

Encapsulation of RNA into LNPs used either Flex S or FlexM (PreciGenome) Systems.

Encapsulation of RNA into LNP with Flex S System:
(organic solution): 40 ul of Lipid Mix
(Aqueous Solution): 160 μl of mRNA in 100 mM sodium acetate (pH:4.0) (mix 32 μg of mRNA with sodium acetate solution to bring final volume to 160 μl)

### Settings:

Flow rate ratio: 4:1 (Aqueous:organic)
Total flow rate: 3 ml/min
N/P ratio: 11.0
Outlet PBS buffer dilution volume equal to total input volume (200 μl for the above example)
   Encapsulation of RNA into LNP FlexM System:
(organic solution): 625 μl of Lipid Mix
(Aqueous Solution): 1875 μl of mRNA in 100 mM sodium acetate (pH:4.0) (mix 500 μg of mRNA with sodium acetate solution to bring final volume to 1875 μl)

### Settings:

Flow rate ratio: 3:1 (Aqueous:organic)
Total flow rate: 3 ml/min
N/P ratio: 11.0

### Buffer Exchange and Filtration of RNA-LNP

After encapsulation is complete, collected samples are filtered and buffer exchange into PBS is performed using Amicon® Ultra-15 Centrifugal Filter Units (30 kDA-100 kDA). Fill the filter unit with ˜14 ml PBS and directly add your sample to the filtration unit. Spin at 1000×g for 20 minutes. Remove the flow-through solution, repeat one more time. Collect nanoparticles in PBS) from the upper compartment of the filter unit.

Flex(M) and Flex(S) samples are prepared two-fold diluted in PBS from their initial formulation volume.

### The Size of LNP Using Dynamice Light Scattering (DLS) System.

The size of Nanoparticles is confirmed using Dynamic Light Scattering (DLS) system. The size of RNA-LNP nanoparticles is usually in the range of 90-130 nM.

### Example 5. MRNA-LNP Delivery to Cells

2 μg of encapsulated mRNA was added per 1×10⁶ 293 cells in a volume of 1 ml. After transfection was completed, the cells were maintained in culture medium at 37° C., 5.0% CO₂. For antibody or protein production, 293 cells were kept at 37° C. with shaking at 200 rpm, and then super was collected at 24-72 hours with secreted antibody or immunostimulant protein for in vitro functional assay.

### Example 6: Sequences of Immunomodulators

To test immunostimulation in vivo with T cells, we transfected with Mix 1 or Mix 2 to tumors. The immunostimulators were either secreted from tumors or were expressed on tumor surface to activate immune cells.

In the examples below, each kind of RNAs was encapsulated into LNPs separately and then they were combined at the ratio of 1:1:1:1.

### A. Mix 1 Containing 4 Immunostimulators.

PMC-2011 GM-CSF
PMC-2024 PD-L1 scFv human Fc (Atezolizumab Ab)
PMC-2124 CXCL-9 TF tag
PMC-2125 IL-12
   6.1 Sequence of GM-CSF DNA Template for In Vitro Transcription of mRNA

Nucleotide sequence of T7AG promoter underlined, then small font 3′UTR, bold capital font GM-CSF sequence; then small front 5′UTR and 152 nucleotide poly A tail

(SEQ ID NO: 2)
TAATACGACTCACTATAAGGAGAAAGCTTacatttgcttctgacacaactgtgttcactagcaacctcaaacag
acaccATGTGGCTTCAGTCCCTGTTGCTCCTCGGAACGGTCGCATGTTCAATAAGC
GCACCTGCGAGAAGTCCGAGTCCTAGCACGCAACCCTGGGAACACGTCAACGC
TATACAGGAGGCGCGACGGCTGCTCAATCTGAGCAGGGACACGGCGGCTGAAA
TGAACGAGACAGTGGAAGTTATCAGCGAAATGTTTGATTTGCAGGAGCCTACG
TGCCTTCAAACAAGGCTTGAACTGTATAAGCAAGGGCTTAGGGGTAGTCTTAC
GAAGCTTAAAGGACCATTGACGATGATGGCTAGTCACTATAAGCAACACTGTC
CACCGACGCCCGAGACGAGTTGCGCTACTCAGATAATCACGTTTGAAAGCTTT
AAAGAGAATCTCAAGGATTTTCTGCTGGTCATTCCTTTCGATTGTTGGGAGCCC
GTGCAAGAGTGATAGTAAGctcgctttcttgctgtccaatttctattaaaggttcctttgttccctaagtccaactactaaact
gggggatattatgaagggccttgagcatctggattctgcctaataaaaaacatttattttcattgcagctcgctttcttgctgtc
caatttctattaaaggttcctttgttccctaagtccaactactaaactgggggatattatgaagggccttgagcatctggattct
gcctaataaaaaacatttattttcattgcaGTCGACTCTAGAAAAAAAAAAAAAAAAAAAAAAAAAAAAAAAAAAAAAAAAA
GGATCCCCGGGCGAGCTCCCAAAAAAAAAAAAAAAAAAAAAAAAAAAAAACCGAA
TTCCTGCAGCTCGAGAAAAAAAAAAAAAAAAAAAAAAAAAAAAAAAAAAAAAAAA
A

### GM-CSF Amino Acid Sequence:

(SEQ ID NO: 3)
MWLQSLLLLGTVACSISAPARSPSPSTQPWEHVNAIQEARRLLNLSRDT
AAEMNETVEVISEMFDLQEPTCLQTRLELYKQGLRGSLTKLKGPLTMMA
SHYKQHCPPTPETSCATQIITFESFKENLKDFLLVIPFDCWEPVQE

All other immunostimulants described below had the same DNA template structure as described in 6.1, except the coding sequence of GM-CSF is replaced.

6.2 Sequence of PD-L1 scFv-Human Fc. Human Fc Underlined. Scfv is from Atezolizumab Antibody in Clinical Use.

Nucleotide sequence of PD-L1
(SEQ ID NO: 4)
ATGGAGACCGACACGCTCCTTCTGTGGGTTCTCCTCCTGTGGGTTCCTGGTTCTACCG
GTGAAGTCCAACTGGTTGAAAGCGGCGGAGGTCTTGTGCAGCCCGGAGGGAGTCTT
AGGTTGTCCTGCGCGGCCTCAGGTTTCACGTTTTCAGACTCTTGGATTCACTGGGTCA
GACAAGCCCCTGGGAAAGGACTTGAGTGGGTGGCATGGATTAGTCCTTATGGGGGA
TCAACTTATTATGCCGACTCTGTTAAAGGCAGGTTTACGATTTCCGCGGACACCTCC
AAAAATACTGCTTACCTTCAGATGAACTCCCTCAGGGCCGAGGATACCGCAGTCTAT
TATTGCGCTAGACGCCATTGGCCAGGCGGTTTTGATTATTGGGGCCAAGGTACCCTC
GTGACGGTATCATCAGGAGGAGGTGGCTCTGGAGGAGGGGGATCTGGCGGAGGAG
GGAGTGATATCCAGATGACACAATCACCAAGCAGTCTGAGTGCCTCCGTGGGTGAC
AGGGTTACTATCACTTGTAGAGCGTCACAGGACGTGAGCACGGCAGTGGCCTGGTA
TCAACAAAAGCCGGGTAAAGCGCCCAAGCTCCTCATTTACTCTGCGAGTTTTCTGTA
TAGTGGAGTGCCTAGTCGGTTCTCTGGTAGCGGGTCAGGTACAGACTTCACTCTGAC
TATATCTAGTTTGCAACCTGAGGATTTCGCAACATATTATTGCCAACAATACTTGTAC
CACCCTGCTACCTTTGGCCAAGGTACAAAAGTAGAGATCAAAAGGACACACACCTG
TCCACCGTGCCCAGCTCCCGAACTGCTTGGGGGCCCGAGTGTTTTTCTTTTCCCTCCT
AAGCCTAAAGATACGCTTATGATCAGCAGGACACCAGAAGTTACTTGTGTGGTAGTT
GATGTGAGCCACGAAGACCCTGAGGTAAAATTTAACTGGTACGTTGACGGAGTAGA
GGTTCATAACGCGAAAACGAAGCCGCGGGAGGAGCAATACAATTCTACATACCGAG
TCGTATCTGTTCTTACAGTCCTCCACCAGGATTGGTTGAACGGCAAGGAGTATAAAT
GCAAAGTGTCTAACAAAGCGCTCCCGGCTCCCATAGAAAAGACCATAAGTAAAGCG
AAGGGTCAACCTCGCGAGCCCCAGGTTTACACTCTCCCTCCATCTAGGGACGAGCTC
ACCAAAAATCAAGTCAGTCTGACATGTCTTGTCAAGGGCTTCTACCCGAGTGATATC
GCAGTGGAGTGGGAGTCCAACGGCCAACCCGAGAACAATTATAAAACAACGCCTCC
CGTGTTGGATAGTGACGGGTCCTTTTTCCTTTACTCAAAATTGACCGTTGACAAGTCA
CGGTGGCAGCAAGGAAACGTTTTTTCTTGCTCTGTAATGCATGAAGCATTGCATAAC
CATTATACCCAGAAAAGCTTGTCTCTCTCTCCGGGCAAGTGATAA
Amino acid sequence:
(SEQ ID NO: 5)
METDTLLLWVLLLWVPGSTGEVQLVESGGGLVQPGGSLRLSCAASGFTFSDSWIHWVR
QAPGKGLEWVAWISPYGGSTYYADSVKGRFTISADTSKNTAYLQMNSLRAEDTAVYYC
ARRHWPGGFDYWGQGTLVTVSSGGGGSGGGGSGGGGSDIQMTQSPSSLSASVGDRVTI
TCRASQDVSTAVAWYQQKPGKAPKLLIYSASFLYSGVPSRFSGSGSGTDFTLTISSLQPE
DFATYYCQQYLYHPATFGQGTKVEIKRTHTCPPCPAPELLGGPSVFLFPPKPKDTLMISR
TPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHE
ALHNHYTQKSLSLSPGK
6.3. CXCL-9 with TF tag (tag underlined)
Nucleotide sequence
(SEQ ID NO: 6)
Atgaagaaaagtggtgttcttttcctcttgggcatcatcttgctggttctgattggagtgcaaggaaccccagtagtgagaaagggtcgctg
ttcctgcatcagcaccaaccaagggactatccacctacaatccttgaaagaccttaaacaatttgccccaagcccttcctgcgagaaaattg
aaatcattgctacactgaagaatggagttcaaacatgtctaaacccagattcagcagatgtgaaggaactgattaaaaagtgggagaaacag
gtcagccaaaagaaaaagcaaaagaatgggaaaaaacatcaaaaaaagaaagttctgaaagttcgaaaatctcaacgttctcgtcaaaagaa
gactacagcggccgcaaaaaacccggatccgtgggegaaaaacctgaacgaaaaagattattaa
Amino acid sequence
(SEQ ID NO: 7)
MKKSGVLFLLGIILLVLIGVQGTPVVRKGRCSCISTNQGTIHLQSLKDLKQFAPSPSCEKIE
IIATLKNGVQTCLNPDSADVKELIKKWEKQVSQKKKQKNGKKHQKKKVLKVRKSQRSR
QKKTTAAAKNPDPWAKNLNEKDY

### 6.4. IL-12

IL-12 is a heterodimer and contained 2 subunits. Nucleotide sequence of IL-12 is shown below: IL-12 beta (p40) with C-terminal Flag tag (bold) underlined, VPGVGVPGVGA linker (italics, SEQ ID NO: 8), IL-12 alpha (p35) (italics, underlined).

(SEQ ID NO: 9)
ATGTGTCATCAGCAGCTCGTTATATCATGGTTTAGTCTTGTTTTTTTGGCGAGTCCGC
TCGTAGCAATTTGGGAACTCAAGAAGGACGTATATGTAGTGGAGCTTGACTGGTATC
CAGACGCCCCTGGTGAAATGGTGGTCTTGACGTGTGACACGCCCGAAGAGGATGGC
ATCACATGGACGCTCGACCAAAGCTCAGAAGTATTGGGGAGCGGCAAGACGCTCAC
TATTCAAGTAAAAGAGTTTGGAGATGCAGGGCAATACACGTGCCACAAAGGAGGAG
AGGTTCTTAGCCATAGTCTGCTCCTGCTGCACAAGAAGGAGGACGGGATCTGGTCCA
CTGATATCCTGAAAGACCAGAAAGAACCCAAGAACAAAACTTTTCTTCGATGTGAA
GCGAAAAATTATTCAGGCCGGTTCACATGCTGGTGGCTTACTACAATATCTACTGAC
CTGACGTTCAGCGTCAAGTCTAGTCGAGGGTCTTCTGATCCGCAAGGTGTCACGTGC
GGGGCCGCCACTCTGTCTGCAGAACGGGTTAGGGGGGACAACAAGGAATATGAATA
TTCCGTGGAGTGCCAAGAAGATAGCGCTTGCCCTGCGGCGGAAGAGAGCCTCCCCA
TCGAGGTTATGGTtGACGCCGTTCATAAACTCAAGTACGAAAACTATACTTCCTCATT
TTTTATAAGGGACATCATAAAGCCGGACCCACCTAAGAATTTGCAATTGAAGCCTTT
GAAGAATAGTgGACAAGTTGAGGTGTCCTGGGAGTACCCGGACACTTGGAGCACTC
CCCATTCATATTTCTCCCTGACTTTCTGCGTTCAGGTTCAGGGCAAATCTAAACGAGA
GAAAAAAGACCGCGTATTCACGGACAAGACTTCTGCCACAGTAATCTGTCGAAAGA
ATGCTTCAATCTCCGTGAGGGCCCAAGACAGATACTATTCATCTAGCTGGTCCGAAT
GGGCCAGTGTTCCGTGCTCAgccgcagactacaaagacgatgacgacaagGTTCCTGGAGTAGGGGT
ACCTGGGGTGGGCgccAGAAACTTGCCAGTTGCGACGCCTGATCCCGGCATGTTTCCTTG
CTTGCACCATTCACAGAATCTGCTTCGAGCTGTCTCCAATATGCTGCAAAAAGCCAGACAA
ACCCTGGAGTTCTACCCATGTACTAGCGAGGAGATAGATCACGAGGATATCACGAAGGAT
AAAACTTCAACAGTCGAAGCTTGTCTTCCCCTGGAGTTGACTAAGAATGAGTCCTGTCTCA
ACTCTCGCGAAACATCTTTCATCACAAATGGTAGCTGTCTCGCGTCTCGGAAAACGTCTTT
CATGATGGCGCTGTGCCTGTCCTCCATATACGAAGACTTGAAAATGTATCAGGTGGAATTC
AAGACCATGAACGCCAAGCTCTTGATGGACCCAAAGAGACAGATCTTTCTTGACCAGAACA
TGCTTGCAGTGATAGATGAGTTGATGCAAGCTTTGAACTTCAACTCAGAAACAGTGCCGCA
AAAATCATCACTTGAGGAGCCTGATTTTTACAAAACGAAAATCAAGTTGTGCATTCTGTTGC
ATGCTTTCCGCATCAGGGCAGTAACTATCGATAGGGTGATGTCCTACCTTAACGCTAGTtaa
Amino acid sequence:
(SEQ ID NO: 10)
MCHQQLVISWFSLVFLASPLVAIWELKKDVYVVELDWYPDAPGEMVVLTCDTPEEDGI
TWTLDQSSEVLGSGKTLTIQVKEFGDAGQYTCHKGGEVLSHSLLLLHKKEDGIWSTDIL
KDQKEPKNKTFLRCEAKNYSGRFTCWWLTTISTDLTFSVKSSRGSSDPQGVTCGAATLS
AERVRGDNKEYEYSVECQEDSACPAAEESLPIEVMVDAVHKLKYENYTSSFFIRDIIKPD
PPKNLQLKPLKNSGQVEVSWEYPDTWSTPHSYFSLTFCVQVQGKSKREKKDRVFTDKT
SATVICRKNASISVRAQDRYYSSSWSEWASVPCSAADYKDDDDKVPGVGVPGVGARNL
PVATPDPGMFPCLHHSQNLLRAVSNMLQKARQTLEFYPCTSEEIDHEDITKDKTSTVEACLPL
ELTKNESCLNSRETSFITNGSCLASRKTSFMMALCLSSIYEDLKMYQVEFKTMNAKLLMDPKR
QIFLDQNMLAVIDELMQALNENSETVPQKSSLEEPDFYKTKIKLCILLHAFRIRAVTIDRVMSYL
NAS

### B. Mix 2 Containing 4 Immunostimulators:

PMC-1893 IL-15 TM
PMC-2067 OX-40 (TF tag)
PMC-2065 CD70 (TF tag)
PMC-2015 PD-1 antibody
   6.5. PMC-1893 IL-15 TM Domain (with CD8 Signaling Peptide and TM Domains)

Nucleotide sequence
(SEQ ID NO: 11)
ATGGCTTTGCCTGTGACTGCACTGTTGCTTCCCTTGGCTCTGCTTCTCCATGCTGCTA
GACCGaactgggtgaatgtaataagtgatttgaaaaaaattgaagatcttattcaatctatgcatattgatgctactttatatacggaa
agtgatgttcaccccagttgcaaagtaacagcaatgaagtgctttctcttggagttacaagttatttcacttgagtccggagatgcaag
tattcatgatacagtagaaaatctgatcatcctagcaaacaacagtttgtcttctaatgggaatgtaacagaatctggatgcaaagaat
gtgaggaactggaggaaaaaaatattaaagaatttttgcagagttttgtGcatattgtccaaatgttcatcaacacttctACaACaACa
CCAGCtCCaaGACCACCAACACCaGCaCCCACCATCGCGTCGCAGCCCCTGTCCCTGCGCCCAGAGGCGT
GCCGGCCAGCGGCGGGGGGCGCAGTGCACACGAGGGGGCTGGACTTCGCCTGTGAT
ATCTACATCTGGGCGCCCCTGGCCGGGACTTGTGGGGTCCTTCTCCTGTCACTGGTTA
TCACCCTTTACTGCtagtgaTAA
Amino acid sequence: CD8 signaling peptide underlined; IL-15 (bold) and CD8
TM in italics
(SEQ ID NO: 12)
MALPVTALLLPLALLLHAARPNWVNVISDLKKIEDLIQSMHIDATLYTESDVHPSCKV
TAMKCFLLELQVISLESGDASIHDTVENLIILANNSLSSNGNVTESGCKECEELEEKN
IKEFLQSFVHIVQMFINTSTTTPAPRPPTPAPTIASQPLSLRPEACRPAAGGAVHTRGLDFA
CDIYIWAPLAGTCGVLLLSLVITLYC

### 6.6. PMC-2067 OX-40

Nucleotide sequence of PMC-2067 OX-40-Tag, TF tag underlined
(SEQ ID NO: 13)
ATGGAGCGCGTACAGCCGCTCGAAGAAAACGTTGGAAATGCAGCCAGACCGCGCTT
TGAGCGGAACAAACTGCTGCTGGTGGCTTCTGTAATTCAGGGTCTTGGCCTGCTGCT
GTGCTTTACCTACATATGTCTTCACTTCTCTGCGCTCCAAGTCAGCCATCGGTATCCA
CGCATTCAATCAATAAAAGTTCAGTTTACTGAATATAAGAAAGAAAAGGGATTCATT
CTGACGAGCCAGAAAGAAGACGAGATCATGAAAGTGCAGAACAACTCTGTTATCAT
AAACTGTGACGGGTTCTACCTCATATCACTCAAAGGTTATTTCTCCCAAGAAGTTAA
CATCAGTCTCCACTACCAAAAAGATGAAGAACCGCTCTTCCAGCTCAAGAAAGTTA
GGTCTGTAAATAGTTTGATGGTCGCTAGTCTTACCTACAAAGATAAGGTGTATTTGA
ACGTAACGACTGATAATACAAGTCTTGATGATTTTCACGTTAACGGAGGCGAATTGA
TACTTATTCATCAGAATCCAGGGGAGTTCTGTGTGCTGAAAAACCCGGATCCGTGGG
CGAAAAACCTGAACGAAAAAGATTATTGA
Amino acid sequence
(SEQ ID NO: 14)
MERVQPLEENVGNAARPRFERNKLLLVASVIQGLGLLLCFTYICLHFSALQVSHRYPRIQSIKVQF
TEYKKEKGFILTSQKEDEIMKVQNNSVIINCDGFYLISLKGYFSQEVNISLHYQKDEEPLFQLKKV
RSVNSLMVASLTYKDKVYLNVTTDNTSLDDFHVNGGELILIHQNPGEFCVLKNPDPWAKNLNE
KDY

### 6.7. PMC-2065 CD70

Nucleotide sequence of PMC-2065 CD70-Tag, TF tag underlined
(SEQ ID NO: 15)
ATGCCCGAAGAAGGGAGCGGTTGTTCTGTGCGAAGGAGACCCTATGGTTGCGTGCT
GCGAGCTGCTCTTGTGCCGTTGGTAGCTGGGTTGGTTATCTGTCTGGTCGTCTGCATT
CAACGCTTCGCGCAAGCGCAACAGCAGCTCCCATTGGAAAGTCTCGGCTGGGACGT
CGCAGAGCTTCAGCTCAACCATACTGGTCCACAGCAAGACCCACGGCTGTACTGGC
AGGGGGGTCCTGCATTGGGGCGCAGTTTCTTGCATGGACCTGAGCTGGATAAAGGTC
AATTGCGCATCCACCGAGATGGCATATACATGGTGCATATCCAGGTCACGCTCGCCA
TTTGCTCTTCAACAACGGCGAGCAGACATCATCCTACGACGCTGGCGGTGGGGATTT
GTTCTCCCGCTAGTCGGTCAATAAGTTTGCTGCGACTCTCTTTTCACCAGGGCTGCAC
AATCGTCAGTCAGAGGCTGACGCCTCTGGCAAGGGGCGATACCCTTTGTACAAACTT
GACCGGGACCCTTCTCCCGAGTAGAAATACCGATGAAACTTTTTTCGGAGTCCAATG
GGTGCGCCCAAAAAACCCGGATCCGTGGGCGAAAAACCTGAACGAAAAAGATTATT
GA
Amino acid
(SEQ ID NO: 16)
MPEEGSGCSVRRRPYGCVLRAALVPLVAGLVICLVVCIQRFAQAQQQLPLESLGWDVA
ELQLNHTGPQQDPRLYWQGGPALGRSFLHGPELDKGQLRIHRDGIYMVHIQVTLAICSS
TTASRHHPTTLAVGICSPASRSISLLRLSFHQGCTIVSQRLTPLARGDTLCTNLTGTLLPSR
NTDETFFGVQWVRPKNPDPWAKNLNEKDY

6.8. PMC-2015 Anti-PD-1 Scfv-Mutant Human Fc with L234A L235A P329G

Nucleotide Sequence
(SEQ ID NO: 17)
ATGGAGACTGATACATTGTTGCTTTGGGTCCTTTTGTTGTGGGTTCCTGGTTCTACGG
GCGCGGCATCACAGGTCCAACTTGTTGAAAGTGGCGGGGGTGTCGTACAGCCCGGA
CGGTCCTTGCGATTGGACTGTAAGGCATCTGGGATTACCTTCTCAAATTCCGGTATG
CATTGGGTTCGCCAAGCCCCGGGCAAGGGTCTCGAATGGGTGGCTGTAATTTGGTAT
GACGGGTCCAAGAGATACTACGCTGATAGTGTGAAGGGTAGATTTACAATATCACG
CGATAATTCAAAAAATACACTGTTTCTGCAGATGAACAGCCTGAGGGCAGAGGACA
CAGCAGTGTATTATTGCGCTACCAATGATGATTATTGGGGGCAGGGTACTCTGGTCA
CTGTTTCAAGTGGTGGTGGTGGTTCAGGTGGTGGGGGATCTGGCGGGGGCGGAAGT
GAGATTGTTCTCACTCAATCACCCGCAACCCTGTCCTTGTCCCCCGGAGAACGCGCG
ACCCTGTCATGCCGCGCTAGTCAATCTGTCAGTAGTTACCTCGCATGGTATCAACAG
AAGCCCGGACAAGCTCCACGGCTCCTCATCTACGATGCCTCAAACAGGGCGACAGG
AATACCCGCACGCTTCTCAGGGTCCGGTAGCGGTACTGACTTTACTTTGACTATATCT
TCCCTCGAGCCGGAAGATTTCGCAGTTTACTATTGCCAACAGTCCAGTAACTGGCCA
AGAACGTTTGGACAAGGAACAAAAGTTGAGATTAAGCGCACCCACACGTGCCCCCC
TTGCCCAGCACCCGAAGCCGCAGGTGGCCCATCAGTGTTTCTTTTTCCTCCAAAACC
AAAAGACACACTCATGATCTCCCGGACGCCTGAGGTGACCTGTGTAGTCGTAGACGT
ATCCCATGAGGACCCTGAAGTAAAGTTTAACTGGTATGTAGACGGTGTGGAAGTAC
ACAATGCCAAGACTAAACCAAGAGAGGAACAGTATAACAGCACCTATAGGGTAGTT
TCCGTGCTCACCGTTCTCCACCAAGATTGGCTTAACGGTAAAGAATATAAATGTAAG
GTGTCAAATAAGGCACTCGGAGCCCCGATCGAAAAGACCATCTCTAAAGCAAAAGG
ACAGCCCAGGGAGCCACAAGTCTACACCCTGCCCCCATCCCGGGATGAGCTGACCA
AGAACCAGGTCAGCCTGACCTGCCTGGTCAAAGGCTTCTATCCCAGCGACATCGCCG
TGGAGTGGGAGAGCAATGGGCAGCCGGAGAACAACTACAAGACCACGCCTCCCGTG
CTGGACTCCGACGGCTCCTTCTTCCTCTACAGCAAGCTCACCGTGGACAAGAGCAGG
TGGCAGCAGGGGAACGTCTTCTCATGCTCCGTGATGCATGAGGCTCTGCACAACCAC
TACACGCAGAAGAGCCTCTCCCTGTCTCCCGGGAAATGA
Amino acid
(SEQ ID NO: 18)
METDTLLLWVLLLWVPGSTGAASQVQLVESGGGVVQPGRSLRLDCKASGITFSNSGMH
WVRQAPGKGLEWVAVIWYDGSKRYYADSVKGRFTISRDNSKNTLFLQMNSLRAEDTA
VYYCATNDDYWGQGTLVTVSSGGGGSGGGGSGGGGSEIVLTQSPATLSLSPGERATLS
CRASQSVSSYLAWYQQKPGQAPRLLIYDASNRATGIPARFSGSGSGTDFTLTISSLEPEDF
AVYYCQQSSNWPRTFGQGTKVEIKRTHTCPPCPAPEAAGGPSVFLFPPKPKDTLMISRTP
EVTCVVVDVSHEDPEVKFNWYVDGVEVHNAKTKPREEQYNSTYRVVSVLTVLHQDWL
NGKEYKCKVSNKALGAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVMHEA
LHNHYTQKSLSLSPGK

### Example 7. The Sequences of Humanized her-2-CD3e Human Fc Bispecific Antibody

The structure of humanized Her-2 ScFv-linker-CD3 ScFv-mutant human Fe is similar to the structure shown in FIG. 2.

Pmc2087 her-2-CD3 Mutant Fe

A DNA template for mRNA in vitro transcription includes T7 promoter (underlined bold below); 5′UTR (regular font), Her-2-CD3-human Fc Ab sequence in bold starts with ATG start codon (underlined) and ends with stop codon TGA (underlined); 3′UTR (regular font); >150 poly A tail with linker in the middle (italics).

(SEQ ID NO: 19)
TAATACGACTCACTATAAGgagaaagcttacatttgcttctgacacaactgtgttcactagcaacctcaaacagacacc
ATGGAGACAGACACACTCCTGCTATGGGTACTGCTGCTCTGGGTTCCAGGTTC
CACTGGCGCCGCTAGCGATATTCAGATGACACAGTCACCGAGCTCCTTGTCTG
CAAGCGTGGGGGACAGGGTTACCATTACTTGCCGGGCATCTCAGGACGTTAAC
ACCGCAGTTGCATGGTACCAGCAGAAGCCCGGTAAAGCACCGAAACTCTTGAT
CTACTCAGCAAGTTTCTTGGAGTCTGGCGTACCAAGTAGATTCAGCGGTTCCAG
ATCAGGTACTGATTTCACGCTTACAATTTCTAGCTTGCAACCCGAGGATTTCGC
GACTTACTACTGCCAGCAACACTATACAACACCCCCTACTTTTGGGCAGGGGAC
TAAAGTCGAGATAAAAGGCGGCGGTGGATCTGGTGGAGGTGGAAGCGGCGGA
GGTGGCTCAGAAGTACAACTTGTTGAGTCCGGTGGTGGACTGGTCCAACCTGG
CGGTTCACTTAGGCTGAGTTGCGCTGCATCAGGTTTTAATATCAAGGACACTTA
CATACATTGGGTCCGGCAGGCTCCAGGAAAAGGACTGGAATGGGTCGCCCGGA
TTTATCCAACCAATGGATATACAAGGTATGCGGATTCAGTTAAAGGAAGATTTA
CTATTTCCGCCGATACGTCCAAAAATACCGCTTACCTCCAGATGAATAGTCTTA
GAGCTGAGGACACCGCCGTCTATTATTGCTCAAGGTGGGGTGGAGATGGGTTT
TACGCTATGGATGTATGGGGGCAGGGCACCCTCGTTACCGTTTCAAGCGGGGG
AGGCGGGTCTGGGGGAGGCGGAAGTGGGGGAGGAGGAAGCGAAGTTCAGCTG
CTCGAATCCGGCGGCGGCCTTGTTCAGCCAGGTGGTAGCTTGAGGCTCAGTTG
TGCTGCATCTGGGTTTACATTCTCAACTTATGCGATGAACTGGGTGAGGCAAGC
ACCTGGAAAGGGACTTGAGTGGGTCTCAAGAATTCGCTCCAAATACAACAACT
ATGCGACGTATTACGCAGACTCAGTGAAAGGACGGTTTACGATATCACGGGAC
GATTCAAAGAATACACTGTATTTGCAGATGAATTCTCTTAGGGCCGAAGACACT
GCCGTATACTATTGTGTACGCCACGGTAATTTTGGCAATAGCTATGTATCTTGG
TTCGCGTACTGGGGCCAAGGCACCCTTGTTACTGTGTCTAGTGGGGGCGGGGG
GAGTGGTGGCGGAGGAAGTGGCGGGGGGGGATCTCAAGCGGTGGTTACTCAA
GAGCCCTCCCTTACTGTTTCTCCGGGCGGGACGGTCACCTTGACTTGTGGCAG
TTCAACAGGGGCAGTCACTACTAGTAATTATGCGAATTGGGTCCAAGAAAAGC
CGGGCCAAGCTTTCCGGGGACTCATCGGAGGAACAAATAAAAGGGCACCCGGC
ACACCCGCGCGCTTTTCCGGGAGTCTTCTGGGCGGCAAGGCAGCCCTCACTCT
CTCTGGGGCTCAACCTGAGGACGAGGCTGAGTACTATTGTGCCCTCTGGTACT
CAAACCTGTGGGTCTTTGGAGGGGGAACCAAGCTTACGGTCTTGTCTAGAGAA
AACCTGTATTTTCAGGGCACCCACACGTGCCCCCCTTGCCCAGCACCCGAAGC
CGCAGGTGGCCCATCAGTGTTTCTTTTTCCTCCAAAACCAAAAGACACACTCAT
GATCTCCCGGACGCCTGAGGTGACCTGTGTAGTCGTAGACGTATCCCATGAGG
ACCCTGAAGTAAAGTTTAACTGGTATGTAGACGGTGTGGAAGTACACAATGCC
AAGACTAAACCAAGAGAGGAACAGTATAACAGCACCTATAGGGTAGTTTCCGT
GCTCACCGTTCTCCACCAAGATTGGCTTAACGGTAAAGAATATAAATGTAAGGT
GTCAAATAAGGCACTCGGAGCCCCGATCGAAAAGACCATCTCTAAAGCAAAAG
GACAGCCCAGGGAGCCACAAGTCTACACCCTGCCCCCATCCCGGGATGAGCTG
ACCAAGAACCAGGTCAGCCTGACCTGCCTGGTCAAAGGCTTCTATCCCAGCGA
CATCGCCGTGGAGTGGGAGAGCAATGGGCAGCCGGAGAACAACTACAAGACC
ACGCCTCCCGTGCTGGACTCCGACGGCTCCTTCTTCCTCTACAGCAAGCTCACC
GTGGACAAGAGCAGGTGGCAGCAGGGGAACGTCTTCTCATGCTCCGTGATGCA
TGAGGCTCTGCACAACCACTACACGCAGAAGAGCCTCTCCCTGTCTCCCGGGA
AATGAgctcgctttcttgctgtccaatttctattaaaggttcctttgttccctaagtccaactactaaactgggggata
ttatgaagggccttgagcatctggattctgcctaataaaaaacatttattttcattgcagctcgctttcttgctgtcca
atttctattaaaggttcctttgttccctaagtccaactactaaactgggggatattatgaagggccttgagcatctgga
ttctgcctaataaaaaacatttattttcattgcaGTCGACTCTAGAAAAAAAAAAAAAAAAAAAAAAAAAAAAAAAAAA
AAAAAAAGGATCCCCGGGCGAGCTCCCAAAAAAAAAAAAAAAAAAAAAAAAAAAAAACCGAATTCCTGCAGCTCGAGAA
AAAAAAAAAAAAAAAAAAAAAAAAAAAAAAAAAAAAAAA

Amino Acid Sequence, her-2-CD3-Mutant Human Fc

Signal peptide underlined, Her-2 Scfv in bold, CD3 Scfv underlined in italics, mutant Fe with mutations in enlarged font and bold.

(SEQ ID NO: 20)
METDTLLLWVLLLWVPGSTGAASDIQMTQSPSSLSASVGDRVTITCRASQDVNTAVA
WYQQKPGKAPKLLIYSASFLESGVPSRFSGSRSGTDFTLTISSLQPEDFATYYCQQH
YTTPPTFGQGTKVEIKGGGGSGGGGSGGGGSEVQLVESGGGLVQPGGSLRLSCAA
SGFNIKDTYIHWVRQAPGKGLEWVARIYPTNGYTRYADSVKGRFTISADTSKNTAY
LQMNSLRAEDTAVYYCSRWGGDGFYAMDVWGQGTLVTVSSGGGGSGGGGSGGGG
SEVQLLESGGGLVQPGGSLRLSCAASGFTESTYAMNWVRQAPGKGLEWVSRIRSKYNNYATYY
ADSVKGRFTISRDDSKNTLYLQMNSLRAEDTAVYYCVRHGNFGNSYVSWFAYWGQGTLVTVSS
GGGGSGGGGSGGGGSQAVVTQEPSLTVSPGGTVTLTCGSSTGAVTTSNYANWVQEKPGQAF
RGLIGGTNKRAPGTPARFSGSLLGGKAALTLSGAQPEDEAEYYCALWYSNLWVFGGGTKLTV
LSRENLYFQGTHTCPPCPAPEAAGGPSVFLFPPKPKDTLMISRTPEVTCVVVDVSHEDPE
VKFNWYVDGVEVHNAKTKPREEQYNSTYRVVSVLTVLHQDWLNGKEYKCKVSNKAL
GAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPG
K
Signaling peptide nucleotide,
SEQ ID NO: 21
ATGGAGACAGACACACTCCTGCTATGGGTACTGCTGCTCTGGGTTCCAGGTTCCACT
GGC
Signaling peptide, amino acid,
SEQ ID NO: 22
METDTLLLWVLLLWVPGSTG
Nucleotide: GCCGCTAGC
Amino Acid: AAS
Her-2 ScFv (VL-linker-VH) nucleotide,
SEQ ID NO: 23
gatattcagatgacacagtcaccgagctccttgtctgcaagcgtgggggacagggttaccattacttgccgggcatctcaggacgttaaca
ccgcagttgcatggtaccagcagaagcccggtaaagcaccgaaactcttgatctactcagcaagtttcttggagtctggcgtaccaagtag
attcagcggttccagatcaggtactgatttcacgcttacaatttctagcttgcaacccgaggatttcgcgacttactactgccagcaacac
tatacaacaccccctacttttgggcaggggactaaagtcgagataaaaggcggcggtggatctggtggaggtggaagcggcggaggtggct
cagaagtacaacttgttgagtccggtggtggactggtccaacctggcggttcacttaggctgagttgcgctgcatcaggttttaatatcaa
ggacacttacatacattgggtccggcaggctccaggaaaaggactggaatgggtcgcccggatttatccaaccaatggatatacaaggtat
gcggattcagttaaaggaagatttactatttccgccgatacgtccaaaaataccgcttacctccagatgaatagtcttagagctgaggaca
ccgccgtctattattgctcaaggtgggggtgagatgggttttacgctatggatgtatgggggcagggcaccctcgttaccgtttcaagc
Her-2 scFv amino acid,
SEQ ID NO: 24
DIQMTQSPSSLSASVGDRVTITCRASQDVNTAVAWYQQKPGKAPKLLIYSASFLESGVPS
RFSGSRSGTDFTLTISSLQPEDFATYYCQQHYTTPPTFGQGTKVEIKGGGGGGGGSGGG
GSEVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDVWGQ
GTLVTVSS
Her-2 VL nucleotide,
SEQ ID NO: 25
Gatattcagatgacacagtcaccgagctccttgtctgcaagcgtgggggacagggttaccattacttgccgggcatctcaggacgttaaca
ccgcagttgcatggtaccagcagaagcccggtaaagcaccgaaactcttgatctactcagcaagtttcttggagtctggcgtaccaagtag
attcagcggttccagatcaggtactgatttcacgcttacaatttctagcttgcaacccgaggatttcgcgacttactactgccagcaacac
tatacaacaccccctacttttgggcaggggactaaagtcgagataaaa
Her-2 VL amino acid,
SEQ ID NO: 26
DIQMTQSPSSLSASVGDRVTITCRASQDVNTAVAWYQQKPGKAPKLLIYSASFLESGVPS
RFSGSRSGTDFTLTISSLQPEDFATYYCQQHYTTPPTFGQGTKVEIK
G4Sx3 linker nucleotide,
SEQ ID NO: 27
GGCGGCGGTGGATCTGGTGGAGGTGGAAGCGGCGGAGGTGGCTCA
G4Sx3 linker amino acid,
SEQ ID NO: 28
GGGGSGGGGSGGGGS
Her-2 VH nucleotide,
SEQ ID NO: 29
Gaagtacaacttgttgagtccggtggtggactggtccaacctggcggttcacttaggctgagttgcgctgcatcaggttttaatatcaagg
acacttacatacattgggtccggcaggctccaggaaaaggactggaatgggtcgcccggatttatccaaccaatggatatacaaggtatgc
ggattcagttaaaggaagatttactatttccgccgatacgtccaaaaataccgcttacctccagatgaatagtcttagagctgaggacacc
gccgtctattattgctcaaggtggggtggagatgggttttacgctatggatgtatgggggcagggcaccctcgttaccgtttcaagc
Her-2 VH amino acid,
SEQ ID NO: 30
EVQLVESGGGLVQPGGSLRLSCAASGFNIKDTYIHWVRQAPGKGLEWVARIYPTNGYT
RYADSVKGRFTISADTSKNTAYLQMNSLRAEDTAVYYCSRWGGDGFYAMDVWGQGT
LVTVSS
G4Sx3 linker nucleotide,
SEQ ID NO: 31
Gggggaggcgggtctgggggaggcggaagtgggggaggaggaagc
G4Sx3 linker amino acid,
SEQ ID NO: 28
GGGGSGGGGSGGGGS
CD3 scFv nucleotide sequence,
SEQ ID NO: 32
Gaagttcagctgctcgaatccggcggcggccttgttcagccaggtggtagcttgaggctcagttgtgctgcatctgggtttacattctcaa
cttatgcgatgaactgggtgaggcaagcacctggaaagggacttgagtgggtctcaagaattcgctccaaatacaacaactatgcgacgta
ttacgcagactcagtgaaaggacggtttacgatatcacgggacgattcaaagaatacactgtatttgcagatgaattctcttagggccgaa
gacactgccgtatactattgtgtacgccacggtaattttggcaatagctatgtatcttggttcgcgtactggggccaaggcacccttgtta
ctgtgtctagtgggggcggggggagtggtggcggaggaagtggcggggggggatctcaagcggtggttactcaagagccctcccttactgt
ttctccgggcgggacggtcaccttgacttgtggcagttcaacaggggcagtcactactagtaattatgcgaattgggtccaagaaaagccg
ggccaagctttccggggactcatcggaggaacaaataaaagggcacccggcacacccgcgcgcttttccgggagtcttctgggcggcaagg
cagccctcactctctctggggctcaacctgaggacgaggctgagtactattgtgccctctggtactcaaacctgtgggtctttggaggggg
aaccaagcttacggtcttg
CD3 scFv amino acid sequence,
SEQ ID NO: 33
EVQLLESGGGLVQPGGSLRLSCAASGFTFSTYAMNWVRQAPGKGLEWVSRIRSKYNNY
ATYYADSVKGRFTISRDDSKNTLYLQMNSLRAEDTAVYYCVRHGNFGNSYVSWFAYW
GQGTLVTVSSGGGGSGGGGSGGGGSQAVVTQEPSLTVSPGGTVTLTCGSSTGAVTTSN
YANWVQEKPGQAFRGLIGGTNKRAPGTPARFSGSLLGGKAALTLSGAQPEDEAEYYCA
LWYSNLWVFGGGTKLTVL
CD3 VH nucleotide,
SEQ ID NO: 34
Gaagttcagctgctcgaatccggcggcggccttgttcagccaggtggtagcttgaggctcagttgtgctgcatctgggtttacattctcaa
cttatgcgatgaactgggtgaggcaagcacctggaaagggacttgagtgggtctcaagaattcgctccaaatacaacaactatgcgacgta
ttacgcagactcagtgaaaggacggtttacgatatcacgggacgattcaaagaatacactgtatttgcagatgaattctcttagggccgaa
gacactgccgtatactattgtgtacgccacggtaattttggcaatagctatgtatcttggttcgcgtactggggccaaggcacccttgtta
ctgtgtctagt
CD3 VH amino acid,
SEQ ID NO: 35
EVQLLESGGGLVQPGGSLRLSCAASGFTFSTYAMNWVRQAPGKGLEWVSRIRSKYNNY
ATYYADSVKGRFTISRDDSKNTLYLQMNSLRAEDTAVYYCVRHGNFGNSYVSWFAYW
GQGTLVTVSS
G4Sx3 linker Nucleotide,
SEQ ID NO: 36
ggg ggc ggg ggg agt ggt ggc gga gga agt ggc ggg ggg gga tct
G4Sx3 linker amino acid,
SEQ ID NO: 28
GGGGSGGGGSGGGGS
CD3 VL nucleotide,
SEQ ID NO: 37
caagcggtggttactcaagagccctcccttactgtttctccgggcgggacggtcaccttgacttgtggcagttcaacaggggcagtcacta
ctagtaattatgcgaattgggtccaagaaaagccgggccaagctttccggggactcateggaggaacaaataaaagggcacccggcacacc
cgcgcgcttttccgggagtcttctgggcggcaaggcagccctcactctctctggggctcaacctgaggacgaggctgagtactattgtgcc
ctctggtactcaaacctgtgggtctttggagggggaaccaagcttacggtcttg
CD3 VL amino acid,
SEQ ID NO: 38
QAVVTQEPSLTVSPGGTVTLTCGSSTGAVTTSNYANWVQEKPGQAFRGLIGGTNKRAPGTPAR
FSGSLLGGKAALTLSGAQPEDEAEYYCALWYSNLWVFGGGTKLTVL
Human Fc with L234AL235AG329G, mutations are shown in bigger font and underlined
Nucleotide,
SEQ ID NO: 39
tctagagaaaacctgtattttcagggcacccacacgtgccccccttgcccagcacccgaagccgcaggtggcccatcagtgtttctttttc
ctccaaaaccaaaagacacactcatgatctcccggacgcctgaggtgacctgtgtagtcgtagacgtatcccatgaggaccctgaagtaaa
gtttaactggtatgtagacggtgtggaagtacacaatgccaagactaaaccaagagaggaacagtataacagcacctatagggtagtttcc
gtgctcaccgttctccaccaagattggcttaacggtaaagaatataaatgtaaggtgtcaaataaggcactcggagccccgatcgaaaaga
ccatctctaaagcaaaaggacagcccagggagccacaagtctacaccctgcccccatcccgggatgagctgaccaagaaccaggtcagcc
tgacctgcctggtcaaaggcttctatcccagcgacatcgccgtggagtgggagagcaatgggcagccggagaacaactacaagaccacg
cctcccgtgctggactccgacggctccttcttcctctacagcaagctcaccgtggacaagagcaggtggcagcaggggaacgtcttctcat
gctccgtgatgcatgaggctctgcacaaccactacacgcagaagagcctctccctgtctcccgggaaatga
Human Fc Amino acid,
SEQ ID NO: 40
SRENLYFQGTHTCPPCPAPEAAGGPSVFLFPPKPKDTLMISRTPEVTCVVVDVSHEDPE
VKFNWYVDGVEVHNAKTKPREEQYNSTYRVVSVLTVLHQDWLNGKEYKCKVSNKAL
GAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPG
K

### Example 8. Sequence of EpCAM-CD3 Human Fc Bispecific Antibody

The structure of the bi-specific EPCAM-CD3-human Fe sequence is shown in FIG. 2. The EpCAM-CD3 human Fe had mutations L234AL235AP329G in Fe to reduce ADCC mediated by Fc. The vector can be either AmpR or Kanamycin resistant for future clinical use. Below is the sequence of EpCAM-CD3 human Fc: T7 AG promoter in front underlined. Coding sequence of EpCAM-CD3 hFc antibody is in bold.

Nucleotide sequence of DNA template to generate mRNA that encodes EpCAM-CD3 human Fc bispecific antibody by in vitro transcription:

(SEQ ID NO: 41)
TAATACGACTCACTATAAGGAGAAAGCTTacatttgcttctgacacaactgtgttcactagcaacctcaaacag
acaccATGGAGACAGACACACTCCTGCTATGGGTACTGCTGCTCTGGGTTCCAGG
TTCCACTGGCGCCGCTAGCCAGGTGCAACTCGTACAAAGCGGTTCCGAACTGA
AGAAACCTGGTGCGAGCGTAAAGGTGTCCTGTAAAGCATCTGGATACACCTTC
ACCAACTACGGAATGAACTGGGTTCGACAGGCACCGGGCCAAGGCTTGGAGTG
GATGGGGTGGATCAACACCTACACGGGTGAACCTACGTACGCAGATGATTTCA
AAGGTCGCTTCGTGTTTTCCCTGGATACTTCTGTGTCCACAGCGTACCTCCAGA
TTTCCAGTCTGAAGGCCGAGGACACAGCCGTATATTACTGTGCTCGGTGGTTG
CGCGATTTTGATTATTGGGGCGCAGGTACTACTGTGACAGTAAGCTCCGGCGG
AGGCGGGTCAGGGGGCGGTGGATCCGGCGGAGGGGGATCAGAAATCGTGCTC
ACTCAATCACCAGCGACCTTGAGCTTGTCACCTGGGGAGCGGGCGACGCTGTC
CTGTAGTGCGTCATCATCTATTTCATATATGCACTGGTATCAACAGAAGCCAGG
ACAGGCGCCTCGCCTGTTGATTTACGATACTAGCAAATTGGCAACGGGGATAC
CCGCTCGATTCAGCGGCAGCGGATCTGGGACGGATTTCACATTGACCATTTCT
AGCCTTGAACCAGAGGACTTCGCAGTATATTACTGCCATCAGAGGTCCAGCTAT
CCATACACATTTGGCGGAGGGACGAAATTGGAAATCAAGGGGGGAGGCGGGT
CTGGGGGAGGCGGAAGTGGGGGAGGAGGAAGCGAAGTTCAGCTGCTCGAATC
CGGCGGCGGCCTTGTTCAGCCAGGTGGTAGCTTGAGGCTCAGTTGTGCTGCAT
CTGGGTTTACATTCTCAACTTATGCGATGAACTGGGTGAGGCAAGCACCTGGA
AAGGGACTTGAGTGGGTCTCAAGAATTCGCTCCAAATACAACAACTATGCGAC
GTATTACGCAGACTCAGTGAAAGGACGGTTTACGATATCACGGGACGATTCAA
AGAATACACTGTATTTGCAGATGAATTCTCTTAGGGCCGAAGACACTGCCGTAT
ACTATTGTGTACGCCACGGTAATTTTGGCAATAGCTATGTATCTTGGTTCGCGT
ACTGGGGCCAAGGCACCCTTGTTACTGTGTCTAGTGGGGGGGGGGGGAGTGGT
GGCGGAGGAAGTGGCGGGGGGGGATCTCAAGCGGTGGTTACTCAAGAGCCCT
CCCTTACTGTTTCTCCGGGCGGGACGGTCACCTTGACTTGTGGCAGTTCAACA
GGGGCAGTCACTACTAGTAATTATGCGAATTGGGTCCAAGAAAAGCCGGGCCA
AGCTTTCCGGGGACTCATCGGAGGAACAAATAAAAGGGCACCCGGCACACCCG
CGCGCTTTTCCGGGAGTCTTCTGGGCGGCAAGGCAGCCCTCACTCTCTCTGGG
GCTCAACCTGAGGACGAGGCTGAGTACTATTGTGCCCTCTGGTACTCAAACCT
GTGGGTCTTTGGAGGGGGAACCAAGCTTACGGTCTTGTCTAGAGAAAACCTGT
ATTTTCAGGGCACCCACACGTGCCCCCCTTGCCCAGCACCCGAAGCCGCAGGT
GGCCCATCAGTGTTTCTTTTTCCTCCAAAACCAAAAGACACACTCATGATCTCC
CGGACGCCTGAGGTGACCTGTGTAGTCGTAGACGTATCCCATGAGGACCCTGA
AGTAAAGTTTAACTGGTATGTAGACGGTGTGGAAGTACACAATGCCAAGACTA
AACCAAGAGAGGAACAGTATAACAGCACCTATAGGGTAGTTTCCGTGCTCACC
GTTCTCCACCAAGATTGGCTTAACGGTAAAGAATATAAATGTAAGGTGTCAAAT
AAGGCACTCGGAGCCCCGATCGAAAAGACCATCTCTAAAGCAAAAGGACAGCC
CAGGGAGCCACAAGTCTACACCCTGCCCCCATCCCGGGATGAGCTGACCAAGA
ACCAGGTCAGCCTGACCTGCCTGGTCAAAGGCTTCTATCCCAGCGACATCGCC
GTGGAGTGGGAGAGCAATGGGCAGCCGGAGAACAACTACAAGACCACGCCTC
CCGTGCTGGACTCCGACGGCTCCTTCTTCCTCTACAGCAAGCTCACCGTGGAC
AAGAGCAGGTGGCAGCAGGGGAACGTCTTCTCATGCTCCGTGATGCATGAGGC
TCTGCACAACCACTACACGCAGAAGAGCCTCTCCCTGTCTCCCGGGAAATGAGc
tcgctttcttgctgtccaatttctattaaaggttcctttgttccctaagtccaactactaaactgggggatattatgaagggccttgagca
tctggattctgcctaataaaaaacatttattttcattgcagctcgctttcttgctgtccaatttctattaaaggttcctttgttccctaag
tccaactactaaactgggggatattatgaagggccttgagcatctggattctgcctaataaaaaacatttattttcattgcaGTCGACTCT
AGAAAAAAAAAAAAAAAAAAAAAAAAAAAAAAAAAAAAAAAAAGGATCCCCGGGCGAGCTCCCA
AAAAAAAAAAAAAAAAAAAAAAAAAAAAACCGAATTCCTGCAGCTCGAGAAAAAA
AAAAAAAAAAAAAAAAAAAAAAAAAAAAAAAAAAA

### Amino Acid Sequence of EpCAM-CD3 Human Fc Bispecific Antibody: (Fc is Shown in Italics, Mutations Underlined)

(SEQ ID NO: 42)
METDTLLLWVLLLWVPGSTGAASQVQLVQSGSELKKPGASVKVSCKASGYTFTNYGM
NWVRQAPGQGLEWMGWINTYTGEPTYADDFKGRFVFSLDTSVSTAYLQISSLKAEDTA
VYYCARWLRDFDYWGAGTTVTVSSGGGGSGGGGSGGGGSEIVLTQSPATLSLSPGERA
TLSCSASSSISYMHWYQQKPGQAPRLLIYDTSKLATGIPARFSGSGSGTDFTLTISSLEPED
FAVYYCHQRSSYPYTFGGGTKLEIKGGGGSGGGGSGGGGSEVQLLESGGGLVQPGGSL
RLSCAASGFTFSTYAMNWVRQAPGKGLEWVSRIRSKYNNYATYYADSVKGRFTISRDD
SKNTLYLQMNSLRAEDTAVYYCVRHGNFGNSYVSWFAYWGQGTLVTVSSGGGGSGG
GGSGGGGSQAVVTQEPSLTVSPGGTVTLTCGSSTGAVTTSNYANWVQEKPGQAFRGLI
GGTNKRAPGTPARFSGSLLGGKAALTLSGAQPEDEAEYYCALWYSNLWVFGGGTKLTV
LSRENLYFQGTHTCPPCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALGAPIEKTIS
KAKGQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
EpCAM VH amino acid sequence
(SEQ ID NO: 43)
QVQLVQSGSELKKPGASVKVSCKASGYTFTNYGMNWVRQAPGQGLEWMGWINTYTG
EPTYADDFKGRFVFSLDTSVSTAYLQISSLKAEDTAVYYCARWLRDFDYWGAGTTVTV
SS
EpCAM VL amino acid sequence
(SEQ ID NO: 44)
EIVLTQSPATLSLSPGERATLSCSASSSISYMHWYQQKPGQAPRLLIYDTSKLATGIPARF
SGSGSGTDFTLTISSLEPEDFAVYYCHQRSSYPYTFGGGTKLEIK

CD3 VH and CD3 CL and human Fc sequences are the same as shown in Example 7.

### Example 9. Sequence of Mesothelin-CD3 Human Fc Bispecific Antibody

The structure of the bi-specific mesothelin-CD3-human Fc sequence is similar to that shown in FIG. 2 except EpCAM Scfv is replaced by mesothelin Scfv.

The mesothelin-CD3 human Fc had mutations L234AL235AP329G in Fc to reduce ADCC mediated by Fc. The vector can be either AmpR or Kanamycin resistant for future clinical use. Below is the nucleoside sequence of mesothelin Scfv-CD3 Scfv human Fc DNA template ending with poly A tail; T7 AG promoter is in the beginning and underlined. Coding sequence of mesothelin-CD3 hFc antibody is in bold.

Nucleotide sequence of template for generating mRNA of mesothelin-CD3 human Fc bispecific antibody by in vitro transcription:

(SEQ ID NO: 45)
TAATACGACTCACTATAAGGAGAAAGCTTacatttgcttctgacacaactgtgttcactagcaacctcaaacag
acaccATGGAGACAGACACACTCCTGCTATGGGTACTGCTGCTCTGGGTTCCAGG
TTCCACTGGCGCCGCTAGCCAGGTACAGCTGCAGCAGTCAGGTCCAGGACTCG
TGACGCCCTCGCAGACCCTCTCACTCACCTGTGCCATCTCCGGGGACAGTGTC
TCTAGCAACAGTGCTACTTGGAACTGGATCAGGCAGTCCCCATCGAGAGGCCT
TGAGTGGCTGGGAAGGACATACTACAGGTCCAAGTGGTATAACGACTATGCAG
TATCTGTGAAAAGTCGAATGAGCATCAACCCAGACACATCCAAGAACCAGTTCT
CCCTGCAGCTGAACTCTGTGACTCCCGAGGACACGGCTGTGTATTACTGTGCA
AGAGGAATGATGACTTACTATTACGGTATGGACGTCTGGGGCCAAGGGACCAC
GGTCACCGTCTCCTCAGGCATTCTAGGATCCGGTGGCGGTGGCAGCGGCGGTG
GTGGTTCCGGAGGCGGCGGTTCTCAGCCTGTGCTGACTCAGTCGTCTTCCCTC
TCTGCATCTCCTGGAGCATCAGCCAGTCTCACCTGCACCTTGCGCAGTGGCATC
AATGTTGGTCCCTACAGGATATACTGGTACCAGCAGAAGCCAGGGAGTCCTCC
CCAGTATCTCCTGAACTACAAATCAGACTCAGATAAGCAGCAGGGCTCTGGAG
TCCCCAGCCGCTTCTCTGGATCCAAAGATGCTTCGGCCAATGCAGGGGTTTTAC
TCATCTCTGGGCTCCGGTCTGAGGATGAGGCTGACTATTACTGTATGATTTGGC
ACAGCAGCGCTGCTGTGTTCGGAGGAGGCACCCAACTGACCGTCCTCTCCGGG
GGAGGCGGGTCTGGGGGAGGCGGAAGTGGGGGAGGAGGAAGCGAAGTTCAGC
TGCTCGAATCCGGCGGCGGCCTTGTTCAGCCAGGTGGTAGCTTGAGGCTCAGT
TGTGCTGCATCTGGGTTTACATTCTCAACTTATGCGATGAACTGGGTGAGGCAA
GCACCTGGAAAGGGACTTGAGTGGGTCTCAAGAATTCGCTCCAAATACAACAA
CTATGCGACGTATTACGCAGACTCAGTGAAAGGACGGTTTACGATATCACGGG
ACGATTCAAAGAATACACTGTATTTGCAGATGAATTCTCTTAGGGCCGAAGACA
CTGCCGTATACTATTGTGTACGCCACGGTAATTTTGGCAATAGCTATGTATCTT
GGTTCGCGTACTGGGGCCAAGGCACCCTTGTTACTGTGTCTAGTGGGGGCGGG
GGGAGTGGTGGCGGAGGAAGTGGCGGGGGGGGATCTCAAGCGGTGGTTACTC
AAGAGCCCTCCCTTACTGTTTCTCCGGGCGGGACGGTCACCTTGACTTGTGGC
AGTTCAACAGGGGCAGTCACTACTAGTAATTATGCGAATTGGGTCCAAGAAAA
GCCGGGCCAAGCTTTCCGGGGACTCATCGGAGGAACAAATAAAAGGGCACCCG
GCACACCCGCGCGCTTTTCCGGGAGTCTTCTGGGCGGCAAGGCAGCCCTCACT
CTCTCTGGGGCTCAACCTGAGGACGAGGCTGAGTACTATTGTGCCCTCTGGTA
CTCAAACCTGTGGGTCTTTGGAGGGGGAACCAAGCTTACGGTCTTGTCTAGAG
AAAACCTGTATTTTCAGGGCACCCACACGTGCCCCCCTTGCCCAGCACCCGAA
GCCGCAGGTGGCCCATCAGTGTTTCTTTTTCCTCCAAAACCAAAAGACACACTC
ATGATCTCCCGGACGCCTGAGGTGACCTGTGTAGTCGTAGACGTATCCCATGA
GGACCCTGAAGTAAAGTTTAACTGGTATGTAGACGGTGTGGAAGTACACAATG
CCAAGACTAAACCAAGAGAGGAACAGTATAACAGCACCTATAGGGTAGTTTCC
GTGCTCACCGTTCTCCACCAAGATTGGCTTAACGGTAAAGAATATAAATGTAAG
GTGTCAAATAAGGCACTCGGAGCCCCGATCGAAAAGACCATCTCTAAAGCAAA
AGGACAGCCCAGGGAGCCACAAGTCTACACCCTGCCCCCATCCCGGGATGAGC
TGACCAAGAACCAGGTCAGCCTGACCTGCCTGGTCAAAGGCTTCTATCCCAGC
GACATCGCCGTGGAGTGGGAGAGCAATGGGCAGCCGGAGAACAACTACAAGA
CCACGCCTCCCGTGCTGGACTCCGACGGCTCCTTCTTCCTCTACAGCAAGCTCA
CCGTGGACAAGAGCAGGTGGCAGCAGGGGAACGTCTTCTCATGCTCCGTGATG
CATGAGGCTCTGCACAACCACTACACGCAGAAGAGCCTCTCCCTGTCTCCCGG
GAAATGAGctcgctttcttgctgtccaatttctattaaaggttcctttgttccctaagtccaactactaaactgggggatattatgaaggg
ccttgagcatctggattctgcctaataaaaaacatttattttcattgcagctcgctttcttgctgtccaatttctattaaaggttcctttgt
tccctaagtccaactactaaactgggggatattatgaagggccttgagcatctggattctgcctaataaaaaacatttattttcattgcaGT
CGACTCTAGAAAAAAAAAAAAAAAAAAAAAAAAAAAAAAAAAAAAAAAAAGGATCCCCGGG
CGAGCTCCCAAAAAAAAAAAAAAAAAAAAAAAAAAAAAACCGAATTCCTGCAGCT
CGAGAAAAAAAAAAAAAAAAAAAAAAAAAAAAAAAAAAAAAAAAA

Amino acid sequence of mesothelin-CD3 human Fc bispecific antibody:

(SEQ ID NO: 46)
METDTLLLWVLLLWVPGSTGAASQVQLQQSGPGLVTPSQTLSLTCAISGDSVSSNSAT
WNWIRQSPSRGLEWLGRTYYRSKWYNDYAVSVKSRMSINPDTSKNQFSLQLNSVTPEDT
AVYYCARGMMTYYYGMDVWGQGTTVTVSSGILGSGGGGSGGGGSGGGGSQPVLTQSSS
LSASPGASASLTCTLRSGINVGPYRIYWYQQKPGSPPQYLLNYKSDSDKQQGSGVPSRES
GSKDASANAGVLLISGLRSEDEADYYCMIWHSSAAVFGGGTQLTVLSGGGGSGGGGS
GGGGSEVQLLESGGGLVQPGGSLRLSCAASGFTFSTYAMNWVRQAPGKGLEWVSRIRSKYN
NYATYYADSVKGRFTISRDDSKNTLYLQMNSLRAEDTAVYYCVRHGNFGNSYVSWFAYWGQG
TLVTVSSGGGGSGGGGSGGGGSQAVVTQEPSLTVSPGGTVTLTCGSSTGAVTTSNYANWVQ
EKPGQAFRGLIGGTNKRAPGTPARFSGSLLGGKAALTLSGAQPEDEAEYYCALWYSNLWVFG
GGTKLTVLSRENLYFQGTHTCPPCPAPEAAGGPSVFLFPPKPKDTLMISRTPEVTCVVVD
VSHEDPEVKFNWYVDGVEVHNAKTKPREEQYNSTYRVVSVLTVLHQDWLNGKEYKC
KVSNKALGAPIEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHEALHNHYTQK
SLSLSPGK
Mesothelin VH amino acid sequence
(SEQ ID NO: 47)
QVQLQQSGPGLVTPSQTLSLTCAISGDSVSSNSATWNWIRQSPSRGLEWLGRTYYRSKWY
NDYAVSVKSRMSINPDTSKNQFSLQLNSVTPEDTAVYYCARGMMTYYYGMDVWGQGTT
VTVSS
Mesothelin VL amino acid sequence
(SEQ ID NO: 48)
QPVLTQSSSLSASPGASASLTCTLRSGINVGPYRIYWYQQKPGSPPQYLLNYKSDSDKQQ
GSGVPSRESGSKDASANAGVLLISGLRSEDEADYYCMIWHSSAAVFGGGTQLTVL

CD3 VH, CD3 VL and human Fc amino acid sequences are the same as those shown in Example 7.

### Example 10. Expression of Immunostimulants or Checkpoint Inhibitors after Transfection of mRNA-LNPs into 293 Cells

The below described immunostimulants mRNA-LNPs were each transfected to HEK-293 cells and expression was confirmed using collected supernatant by either FACS, Western blotting or ELISA. GM-CSF protein expression was detected by ELISA after transfection of mRNA.

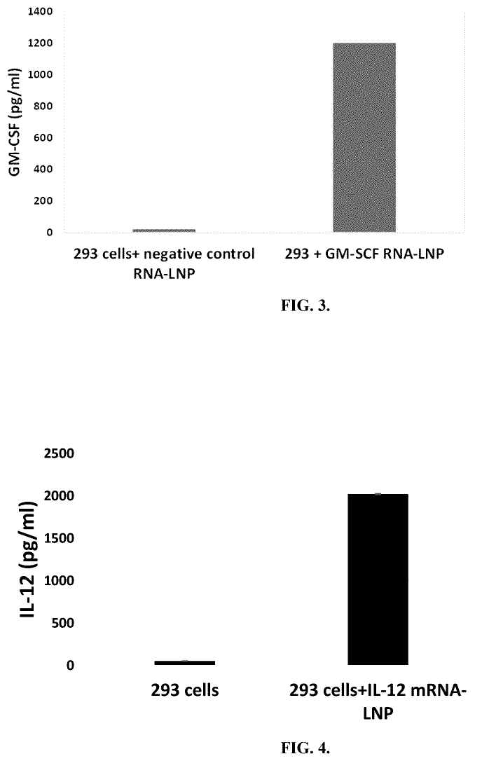

Similarly, IL-12 expression in collected supernatant after transfection IL-12 mRNA-LNP (PMC2125) to HEK-293 cells was shown on FIG. 4.

Also, PMC2015 mRNA-LNP encoding PD-1 Scfv hFc was transfected to HEK-293 cells. Expression of anti-PD-1 scFv-hFc after transfecting 293 cells was detected on cell surface by FACS.

Similarly, mRNA-LNP encoding anti-PD-L1 Scfv hFc was transfected to HEK-293 cells. Expression of anti-PD-L1 scFv-hFc after transfecting HEK-293 cells was detected on cell surface by FACS.

### Example 11. Activation of Immune Cell Expansion with Immunostimulants

Increased NK cell expansion was observed after transfecting IL-12 mRNA-LNP into NK cells, which shows functional activity of immunostimulant IL-12 (FIG. 5). NK cells control without IL-12 did not expand and died in the medium, while NK cells expanded with IL-12 secreted into the medium after transfecting mRNA-LNP (FIG. 5). The results validates functional effect of IL-12 mRNA-LNP on activation of immune cells in vitro.

### Example 12. Activation of Tumor Cell Killing In Vitro with mRNA-LNP Immunostimulants, Bispecific EpCam-CD3 Antibody and PBMC Cells

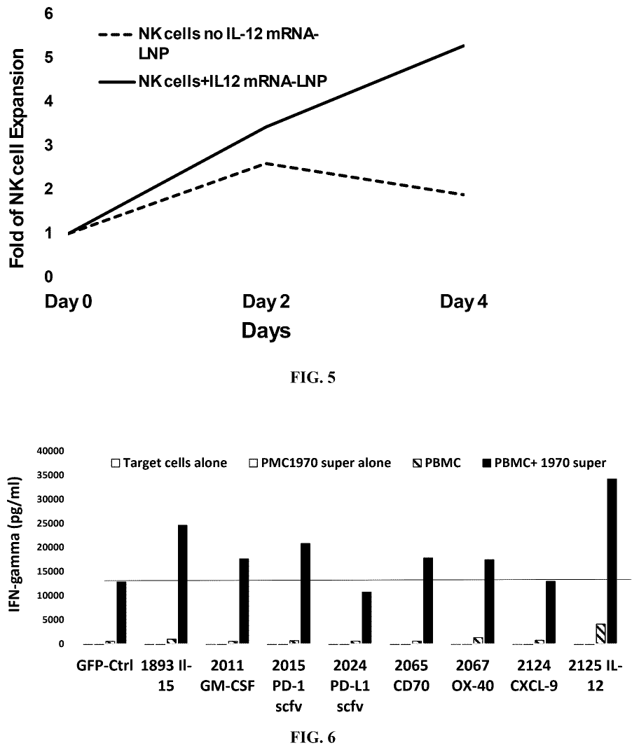

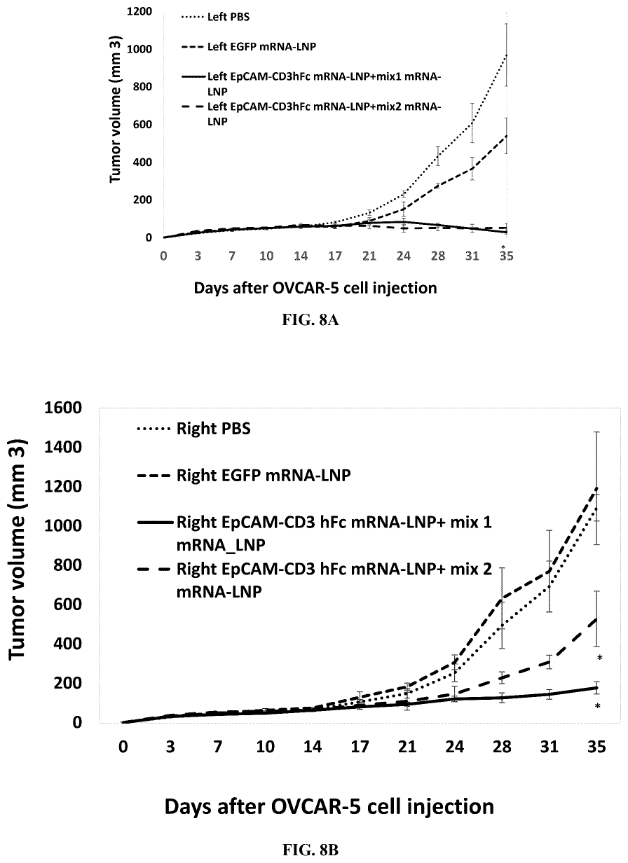

To test the effect of immunostimulants delivered into tumor cells, different mRNA-LNPs were transfected to colorectal OVCAR-5 cells (FIG. 6). Then bispecific EpCAM-CD3 antibody and PBMC cells were used in cytotoxicity assay and IFN-gamma assay either with OVCAR-5 cells transfected with negative control GFP mRNA-LNP, or with OVCAR-5 cells transfected with different immunostimulant mRNA-LNPs (FIG. 8). Immunostimulants such as IL-12, IL-15-TM, PD-1 scfv-hFc, CD70, and OX-40 increased IFN-gamma secretion (FIG. 6) in OVCAR-5 cells, and induced cytotoxicity mediated by EpCAM-CD3 mRNA-LNP and PBMC (data not shown).

### Example 13. Increased Efficacy by Immunostimulant mRNA-LNP and EpCAM-CD3 mRNA-LNP in OVCAR-5 Xenograft Mouse Study In Vivo

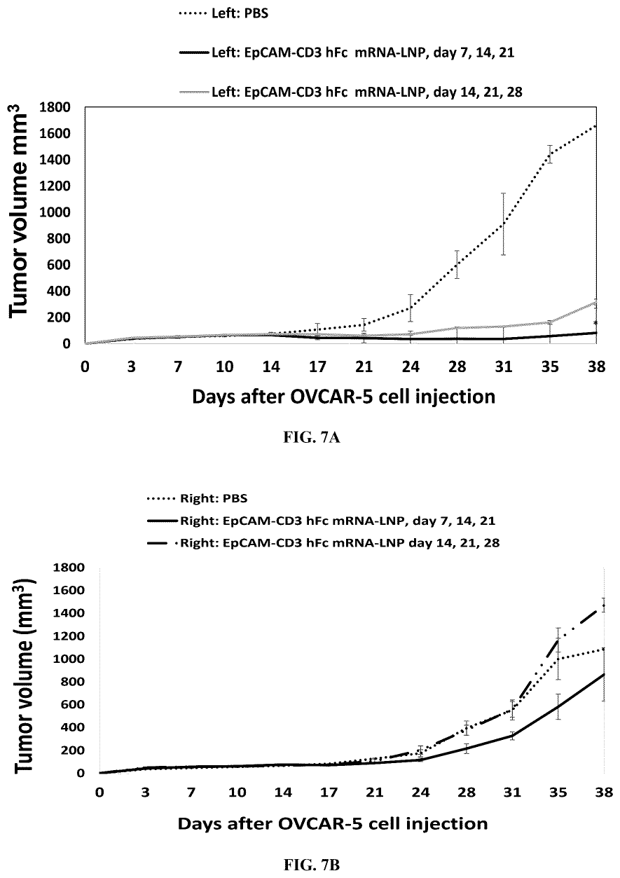

To test in vivo effect of immunostimulants, OVCAR-5 xenograft NSG mouse model was used with EpCAM-CD3-hFc mRNA-LNP and T cells (FIG. 7). The OVCAR-5 tumor cells were injected to both left and right flanks to grow tumors. Then 1 microgram of bispecific antibody EpCAM-Cd3-hFc mRNA-LNP was intratumorally injected only to the left side tumors either at days 7, 14, 21 or days 14, 21, 28, and then 1×10⁷ T cells per mice were injected intravenously on day 9. EpCAM-CD3 hFc mRNA-LNP significantly decreased xenograft tumor size and volume on the injected left side (FIG. 7A) but not on the non-injected right side (FIG. 7B). It shows that without immunostimulant activation of T cells in vivo, EpCAM-CD3 antibody was secreted from tumors and only had local efficacy around tumors.

### Example 14. Four-Immunostimulant Mix mRNA-LNP Decreased Tumor Growth Treated with EpCAM-CD3-hFc mRNA-LNP and T Cells

This experiment also contained OVCAR-5 xenograft tumor cells injected to the left and right sides, and then EpCAM-CD3 mRNA-LNP with immunostimulant mRNA-LNP were injected into the left side only to observe tumor growth on distant untreated right tumor side.

In this experiment, immunostimulant mRNA-LNP cocktails 1 and 2 (each cocktail mix contained four different immunostimulants) were added next day after EpCAM-CD3-hFc RNA-LNP intratumoral administration. Then T cells were injected by i.v. 2 days after EpCAM-CD3hFc mRNA-LNP injection. The EpCAM-CD3 hFc mRNA-LNP were injected 3 times into the tumors weekly starting at day 14. Mix 1 and mix 2 contained the following immunostimulants:

Mix 1 Contained 4 Immunostimulators: All mRNA-LNP were Mixed at Ratio 1:1:1:1.
PMC-2011 GM-CSF
PMC-2024 PD-L1 scFv human Fe (Atezolizumab Ab)
PMC-2124 CXCL-9 TF tag
PMC-2125 IL-12
   Mix 2 Contained 4 Immunostimulators: All mRNA-LNP were Mixed at Ratio 1:1:1:1.
PMC-1893 IL-15 TM
PMC-2067 OX-40 (TF tag)
PMC-2065 CD70 (TF tag)
PMC-2015 PD-1 antibody

In the presence of immunostimulant mRNA-LNP cocktails, EpCAM-CD3 hFc mRNA-LNP significantly decreased OVCAR-5 xenograft tumor growth at the treated left side (FIG. 8A) and also at distant untreated right side (FIG. 8B). The distant right side with tumor cells can be a model for metastatic disseminated cells. The results demonstrate that Mix 1 decreased distant tumor growth better than Mix 2.

### Example 15. Immunostimulant Mix mRNA-LNPs (GMCSF+IL-12) Decreased Tumor Growth Treated with EpCAM-CD3-hFc mRNA-LNP and T Cells at Both Local and Distant Side

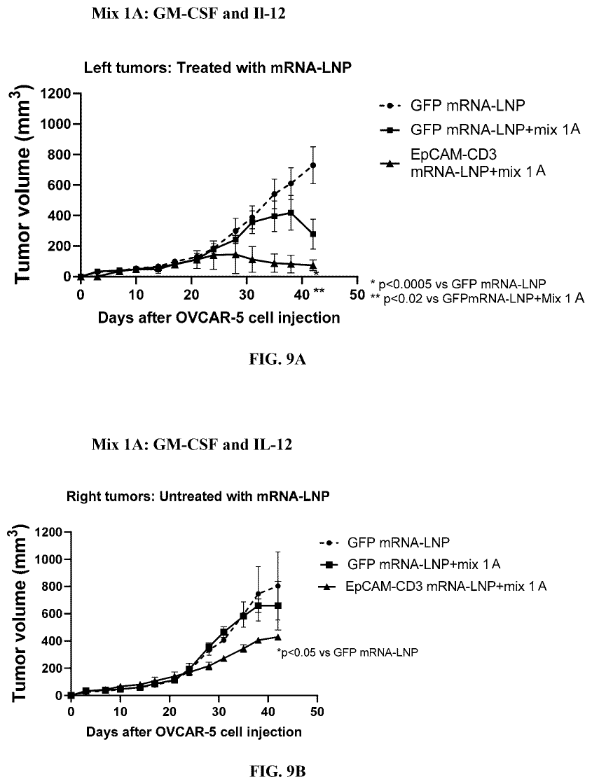

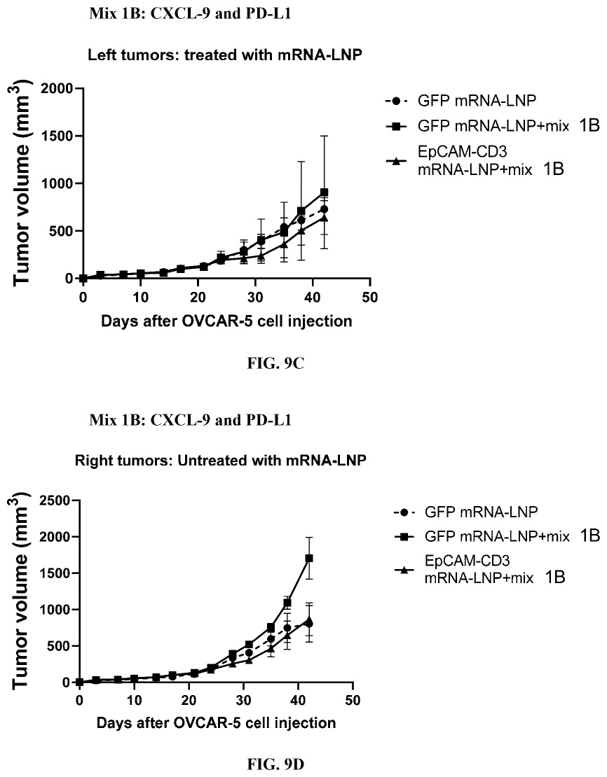

To narrow down the number of immunostimulants to two, the immunostimulant Mix 1 of Example 14 was divided to Mix 1A: GM-CSF (PMC2011) and IL-12 (PMC2125) and Mix 1B: CXCL-9 (PMC2124) and PD-L1 (PMC2024) in this experiment. In addition, PBMC rather than T cells were injected to mice in order to include other immune cell type to provide additional stimulating signaling (FIGS. 9A and 9B). The 2×10⁶ OVCAR-5 cells were injected to both sides subcutaneously, and then either GFP mRNA or EpCAM-CD3 hFc mRNA (1 μg/mice) were injected intratumorally on days 14, 21 and 28 to the left side tumors. Mix 1A mRNA-LNP or Mix 1B mRNA-LNPs (1 μg/mice) was injected to the left side tumors on days 15, 22 and 29. Then PBMC cells (1×10⁷ cells/mice) were injected by iv on days 16, 23 and 30. EpCAM-CD3 hFc mRNA-LNP plus Mix 1A immunostimulants (IL-12+GM-CSF) mRNA-LNP significantly decreased tumor growth not only on the local left side (FIG. 9A) but also on the right distant side (FIG. 9B). The EpCAM-CD3hFC with Mix 1B (CXCL-9+PD-L1) mRNA-LNP did not decrease tumor growth on the local left (FIG. 9C) or distant right side (FIG. 9D).

### Example 16. Immunostimulant Mix mRNA-LNPs (GMCSF+IL-12) Decreased Tumor Growth Treated with EpCAM-CD3-hFc mRNA-LNP and T Cells at Both Local and Distant Side

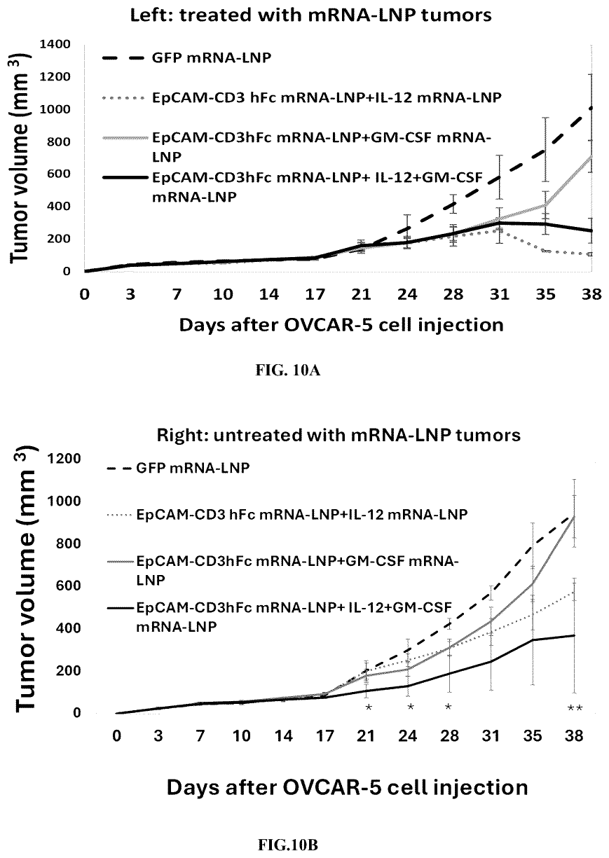

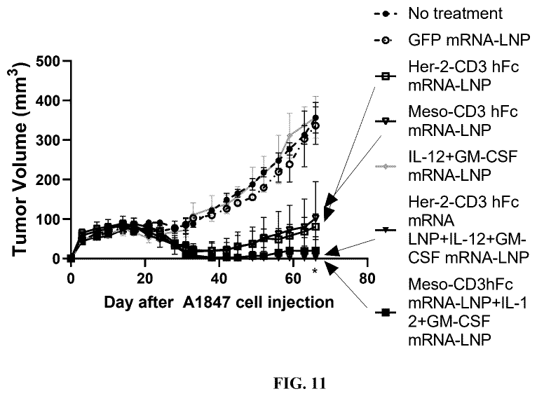

In this experiment, IL-12 mRNA-LNP alone or GM-CSF mRNA-LNP alone was compared to GM-CSF+IL-12 mRNA-LNP sub-Mix 1A to test whether one immunostimulant is enough or whether the combination is better (FIG. 10). The left side was injected with 2×10⁶ OVCAR-5 cells, and the right side was injected with 1×10⁶ cells subcutaneously. Then EpCAM-CD3 mRNA-LNP was injected to the left side tumors at days 14, 21 and 28. mRNA-LNP of IL-12, GM-CSF, or IL-12+GM-CSF mix was injected to the left tumors at days 15, 22 and 29. Then 1×10⁷ of PBMC cells per mice were injected intravenously on days 16, 23 and 30. GM-CSF alone did not have a significant effect on decreasing distant tumors. IL-12+GM-CSF mRNA-LNP with EpCAM-CD3 hFc mRNA-LNP and PBMC was more effective than IL-12 or GM-CSF mRNA-LNP alone at reducing distant tumor growth at days 21-28 and was less toxic that IL-12 alone (FIGS. 10A and 10B).

The results show that tumors treated with EPCAM and CD3e bispecific antibody mRNA-LNP intratumorally were destroyed in the presence of T or PBMC cells, and the destroyed tumor cells serve as a natural vaccine, presenting to immune cells neoantigens or tumor-specific antigens. In the presence of immunostimulant mRNA-LNPs which activate immune cells, distant cells can be targeted and killed. The distant site tumor cells were destroyed by activated T/PBMC cells. This is an effective approach for targeting primary and metastatic tumors. It has advantage versus chemotherapy that stimulates immune cells because it brings to the tumor site T cells and immune cells which have memory function and thus provide durable effect.

### Example 17. Combination of her-2-CD3 and Mesothelin-CD3hFc mRNA-LNP with GM-CSF and IL-12 mRNA Decreased Tumor Growth In Vivo

In this experiment, we performed intratumoral treatment of ovarian A1847 xenograft tumors with (i) mesothelin-CD3hFc and IL-12+GM-CSF mRNA-LNP, and (ii) Her-2-CD3hFc mRNA-LNP and IL-12+GM-CSF mRNA-LNP. The experimental protocols were similar to those described in Example 13.

Her-2 and Mesothelin are extracellular proteins that are often overexpressed in many types of solid tumors and they are often used for targeting with antibodies or CAR-T cell therapy.

As shown in FIG. 11, both mRNA-LNP Her-2-CD3hFc and mRNA-LNP Mesothelin-CD3 hFc in combination with mRNA-LNP IL-12 and mRNA-LNP GM-CSF significantly decreased tumor growth locally more than each antibody alone without the stimulant. IL-12 mRNA-LNP and GM-CSF mRNA-LNP alone did not decrease tumor growth in this A1847 xenograft in vivo model. The results show increased efficacy of combination of bispecific antibody with immunostimulant (GM-CSF and IL-12) mRNA-LNP versus treating with bispecific antibody mRNA-LNP alone.

What is claimed is:

1. A method for treating cancer, comprising the steps of:
obtaining first lipid nanoparticles (LNPs) encapsulating a first mRNA that encodes a bispecific antigen-binding molecule comprising a first ScFv and CD3e ScFv, wherein said first scFv is against EPCAM, Her-2, Mesothelin, EGFR, PLAP, CD147, 4-1BB, c-Met, CD19/CD37, PSMA, CD47, CD19, BCMA, or Claudin 18.2;
obtaining second LNPs encapsulating a second mRNA that encodes GM-CSF;
obtaining third LNPs encapsulating a third mRNA that encodes IL-12; and
administering the first, the second, and the third mRNA-encapsulated LNPs s to a tumor lesion of a subject having cancer.

2. The method of claim 1, wherein the cancer is metastatic solid cancer.

3. The method of claim 1, which activates T cells and/or NK cells in the tumor microenvironment to treat tumor at the administered site and tumor at a distant site.

4. The method of claim 1, wherein the bispecific antigen-binding molecule comprises a monovalent humanized anti-EPCAM ScFv and a monovalent CD3e ScFv fused to human Fc, wherein the Fc is optionally mutated.

5. The method of claim 1, wherein the bispecific antigen-binding molecule comprises a monovalent humanized anti-Her-2 ScFv and a monovalent CD3e ScFv fused to human Fc, wherein the Fc is optionally mutated.

6. The method of claim 1, wherein the bispecific antigen-binding molecule comprises a monovalent humanized anti-mesothelin ScFv and a monovalent CD3e ScFv fused to human Fc, wherein the Fe is optionally mutated.

7. The method of claim 1, wherein the first, the second, and the third LNPs are administered to the subject simultaneously.

8. The method of claim 1, wherein the first, the second, and the third LNPs are administered to the subject sequentially.

9. The method of claim 1, wherein the first mRNA is transcribed from a DNA sequence comprising: (a) a promoter coding sequence, (b) 5′-UTR (untranslated region) coding sequence, (c) a sequence encoding the bispecific antigen-binding molecule, (d) a 3′-UTR coding sequence, and (e) a poly A tail sequence.

10. The method of claim 1, wherein the second mRNA is transcribed from a DNA sequence comprising: (a) a promoter coding sequence, (b) 5′-UTR (untranslated region) coding sequence, (c) a sequence encoding GM-CSF, (d) a 3′-UTR coding sequence, and (e) a poly A tail sequence.

11. The method of claim 1, wherein the third mRNA is transcribed from a DNA sequence comprising: (a) a promoter coding sequence, (b) 5′-UTR (untranslated region) coding sequence, (c) a sequence encoding IL-12, (d) a 3′-UTR coding sequence, and (e) a poly A tail sequence.

12. The method of claim 1, wherein said cancer is colorectal, ovarian, pancreatic, breast, or lung cancer.

13. The method of claim 1, wherein the LNPs comprise 8-[(2-hydroxyethyl)[6-oxo-6-(undecyloxy)hexyl]amino]-octanoic acid, 1-octylnonyl ester (SM-102), distearoylphosphatidylcholine (DSPC), Cholesterol, and 1,2-dimyristoyl-rac-glycero-3-methoxypolyethylene glycol-2000 (DMG-PEG2000).

14. A method for treating cancer, comprising the steps of:
obtaining lipid nanoparticles (LNPs) encapsulating (a) a first mRNA that encodes a bispecific antigen-binding molecule comprising a first ScFv and CD3e ScFv, wherein said first scFv is against EPCAM, Her-2, Mesothelin, EGFR, PLAP, CD147, 4-1BB, c-Met, CD19/CD37, PSMA, CD47, CD19, BCMA, Claudin 18.2, (b) a second mRNA that encodes Gm-CSF, and (c) a third mRNA that encodes IL-12;
administering the mRNA-encapsulated LNPs to a tumor lesion of a subject having cancer.

15. The method of claim 14, wherein the cancer is metastatic solid cancer.

16. The method of claim 14, which activates T cells and/or NK cells in the tumor microenvironment to treat tumor at the administered site and tumor at a distant site.

17. The method of claim 14, wherein the bispecific antigen-binding molecule comprises a monovalent humanized anti-EPCAM ScFv and a monovalent CD3e ScFv fused to human Fc, wherein the Fc is optionally mutated.

18. The method of claim 14, wherein the bispecific antigen-binding molecule comprises a monovalent humanized anti-Her-2 ScFv and a monovalent CD3e ScFv fused to human Fc, wherein the Fc is optionally mutated.

19. The method of claim 14, wherein the bispecific antigen-binding molecule comprises a monovalent humanized anti-mesothelin ScFv and a monovalent CD3e ScFv fused to human Fe, wherein the Fe is optionally mutated.

20. The method of claim 14, wherein the LNPs comprise 8-[(2-hydroxyethyl)[6-oxo-6-(undecyloxy)hexyl]amino]-octanoic acid, 1-octylnonyl ester (SM-102), distearoylphosphatidylcholine (DSPC), Cholesterol, and 1,2-dimyristoyl-rac-glycero-3-methoxypolyethylene glycol-2000 (DMG-PEG2000).


SEQUENCE LISTING (48 sequences)

SEQ ID NO: 1 - protein, 5 aa, synthetic construct
GGGGS

SEQ ID NO: 2 - DNA, 949 bp, Homo sapiens
  source 1..949: mol_type = genomic DNA
taatacgactcactataaggagaaagcttacatttgcttctgacacaactgtgttcactagcaacctcaaacagacaccatgtggcttcagtccctgttgctcctcggaacggtcgcatgttcaataagcgcacctgcgagaagtccgagtcctagcacgcaaccctgggaacacgtcaacgctatacaggaggcgcgacggctgctcaatctgagcagggacacggcggctgaaatgaacgagacagtggaagttatcagcgaaatgtttgatttgcaggagcctacgtgccttcaaacaaggcttgaactgtataagcaagggcttaggggtagtcttacgaagcttaaaggaccattgacgatgatggctagtcactataagcaacactgtccaccgacgcccgagacgagttgcgctactcagataatcacgtttgaaagctttaaagagaatctcaaggattttctgctggtcattcctttcgattgttgggagcccgtgcaagagtgatagtaagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcagtcgactctagaaaaaaaaaaaaaaaaaaaaaaaaaaaaaaaaaaaaaaaaaggatccccgggcgagctcccaaaaaaaaaaaaaaaaaaaaaaaaaaaaaaccgaattcctgcagctcgagaaaaaaaaaaaaaaaaaaaaaaaaaaaaaaaaaaaaaaaaa

SEQ ID NO: 3 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 4 - protein, 1464 aa, Homo sapiens
ATGGAGACCGACACGCTCCTTCTGTGGGTTCTCCTCCTGTGGGTTCCTGGTTCTACCGGTGAAGTCCAACTGGTTGAAAGCGGCGGAGGTCTTGTGCAGCCCGGAGGGAGTCTTAGGTTGTCCTGCGCGGCCTCAGGTTTCACGTTTTCAGACTCTTGGATTCACTGGGTCAGACAAGCCCCTGGGAAAGGACTTGAGTGGGTGGCATGGATTAGTCCTTATGGGGGATCAACTTATTATGCCGACTCTGTTAAAGGCAGGTTTACGATTTCCGCGGACACCTCCAAAAATACTGCTTACCTTCAGATGAACTCCCTCAGGGCCGAGGATACCGCAGTCTATTATTGCGCTAGACGCCATTGGCCAGGCGGTTTTGATTATTGGGGCCAAGGTACCCTCGTGACGGTATCATCAGGAGGAGGTGGCTCTGGAGGAGGGGGATCTGGCGGAGGAGGGAGTGATATCCAGATGACACAATCACCAAGCAGTCTGAGTGCCTCCGTGGGTGACAGGGTTACTATCACTTGTAGAGCGTCACAGGACGTGAGCACGGCAGTGGCCTGGTATCAACAAAAGCCGGGTAAAGCGCCCAAGCTCCTCATTTACTCTGCGAGTTTTCTGTATAGTGGAGTGCCTAGTCGGTTCTCTGGTAGCGGGTCAGGTACAGACTTCACTCTGACTATATCTAGTTTGCAACCTGAGGATTTCGCAACATATTATTGCCAACAATACTTGTACCACCCTGCTACCTTTGGCCAAGGTACAAAAGTAGAGATCAAAAGGACACACACCTGTCCACCGTGCCCAGCTCCCGAACTGCTTGGGGGCCCGAGTGTTTTTCTTTTCCCTCCTAAGCCTAAAGATACGCTTATGATCAGCAGGACACCAGAAGTTACTTGTGTGGTAGTTGATGTGAGCCACGAAGACCCTGAGGTAAAATTTAACTGGTACGTTGACGGAGTAGAGGTTCATAACGCGAAAACGAAGCCGCGGGAGGAGCAATACAATTCTACATACCGAGTCGTATCTGTTCTTACAGTCCTCCACCAGGATTGGTTGAACGGCAAGGAGTATAAATGCAAAGTGTCTAACAAAGCGCTCCCGGCTCCCATAGAAAAGACCATAAGTAAAGCGAAGGGTCAACCTCGCGAGCCCCAGGTTTACACTCTCCCTCCATCTAGGGACGAGCTCACCAAAAATCAAGTCAGTCTGACATGTCTTGTCAAGGGCTTCTACCCGAGTGATATCGCAGTGGAGTGGGAGTCCAACGGCCAACCCGAGAACAATTATAAAACAACGCCTCCCGTGTTGGATAGTGACGGGTCCTTTTTCCTTTACTCAAAATTGACCGTTGACAAGTCACGGTGGCAGCAAGGAAACGTTTTTTCTTGCTCTGTAATGCATGAAGCATTGCATAACCATTATACCCAGAAAAGCTTGTCTCTCTCTCCGGGCAAGTGATAA

SEQ ID NO: 5 - protein, 486 aa, Homo sapiens
METDTLLLWVLLLWVPGSTGEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - DNA, 432 bp, Homo sapiens
  source 1..432: mol_type = genomic DNA
atgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacagcggccgcaaaaaacccggatccgtgggcgaaaaacctgaacgaaaaagattattaa

SEQ ID NO: 7 - protein, 143 aa, Homo sapiens
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTTAAAKNPDPWAKNLNEKDY

SEQ ID NO: 8 - protein, 11 aa, synthetic construct
VPGVGVPGVGA

SEQ ID NO: 9 - DNA, 1641 bp, Homo sapiens
  source 1..1641: mol_type = genomic DNA
atgtgtcatcagcagctcgttatatcatggtttagtcttgtttttttggcgagtccgctcgtagcaatttgggaactcaagaaggacgtatatgtagtggagcttgactggtatccagacgcccctggtgaaatggtggtcttgacgtgtgacacgcccgaagaggatggcatcacatggacgctcgaccaaagctcagaagtattggggagcggcaagacgctcactattcaagtaaaagagtttggagatgcagggcaatacacgtgccacaaaggaggagaggttcttagccatagtctgctcctgctgcacaagaaggaggacgggatctggtccactgatatcctgaaagaccagaaagaacccaagaacaaaacttttcttcgatgtgaagcgaaaaattattcaggccggttcacatgctggtggcttactacaatatctactgacctgacgttcagcgtcaagtctagtcgagggtcttctgatccgcaaggtgtcacgtgcggggccgccactctgtctgcagaacgggttaggggggacaacaaggaatatgaatattccgtggagtgccaagaagatagcgcttgccctgcggcggaagagagcctccccatcgaggttatggttgacgccgttcataaactcaagtacgaaaactatacttcctcattttttataagggacatcataaagccggacccacctaagaatttgcaattgaagcctttgaagaatagtggacaagttgaggtgtcctgggagtacccggacacttggagcactccccattcatatttctccctgactttctgcgttcaggttcagggcaaatctaaacgagagaaaaaagaccgcgtattcacggacaagacttctgccacagtaatctgtcgaaagaatgcttcaatctccgtgagggcccaagacagatactattcatctagctggtccgaatgggccagtgttccgtgctcagccgcagactacaaagacgatgacgacaaggttcctggagtaggggtacctggggtgggcgccagaaacttgccagttgcgacgcctgatcccggcatgtttccttgcttgcaccattcacagaatctgcttcgagctgtctccaatatgctgcaaaaagccagacaaaccctggagttctacccatgtactagcgaggagatagatcacgaggatatcacgaaggataaaacttcaacagtcgaagcttgtcttcccctggagttgactaagaatgagtcctgtctcaactctcgcgaaacatctttcatcacaaatggtagctgtctcgcgtctcggaaaacgtctttcatgatggcgctgtgcctgtcctccatatacgaagacttgaaaatgtatcaggtggaattcaagaccatgaacgccaagctcttgatggacccaaagagacagatctttcttgaccagaacatgcttgcagtgatagatgagttgatgcaagctttgaacttcaactcagaaacagtgccgcaaaaatcatcacttgaggagcctgatttttacaaaacgaaaatcaagttgtgcattctgttgcatgctttccgcatcagggcagtaactatcgatagggtgatgtcctaccttaacgctagttaa

SEQ ID NO: 10 - protein, 546 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSGQVEVSWEYPDTWSTPHSYFSLTFCVQVQGKSKREKKDRVFTDKTSATVICRKNASISVRAQDRYYSSSWSEWASVPCSAADYKDDDDKVPGVGVPGVGARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 11 - DNA, 621 bp, Homo sapiens
  source 1..621: mol_type = genomic DNA
atggctttgcctgtgactgcactgttgcttcccttggctctgcttctccatgctgctagaccgaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtgcatattgtccaaatgttcatcaacacttctacaacaacaccagctccaagaccaccaacaccagcacccaccatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgctagtgataa

SEQ ID NO: 12 - protein, 204 aa, Homo sapiens
MALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 13 - DNA, 597 bp, Homo sapiens
  source 1..597: mol_type = genomic DNA
atggagcgcgtacagccgctcgaagaaaacgttggaaatgcagccagaccgcgctttgagcggaacaaactgctgctggtggcttctgtaattcagggtcttggcctgctgctgtgctttacctacatatgtcttcacttctctgcgctccaagtcagccatcggtatccacgcattcaatcaataaaagttcagtttactgaatataagaaagaaaagggattcattctgacgagccagaaagaagacgagatcatgaaagtgcagaacaactctgttatcataaactgtgacgggttctacctcatatcactcaaaggttatttctcccaagaagttaacatcagtctccactaccaaaaagatgaagaaccgctcttccagctcaagaaagttaggtctgtaaatagtttgatggtcgctagtcttacctacaaagataaggtgtatttgaacgtaacgactgataatacaagtcttgatgattttcacgttaacggaggcgaattgatacttattcatcagaatccaggggagttctgtgtgctgaaaaacccggatccgtgggcgaaaaacctgaacgaaaaagattattga

SEQ ID NO: 14 - protein, 198 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLKNPDPWAKNLNEKDY

SEQ ID NO: 15 - DNA, 627 bp, Homo sapiens
  source 1..627: mol_type = genomic DNA
atgcccgaagaagggagcggttgttctgtgcgaaggagaccctatggttgcgtgctgcgagctgctcttgtgccgttggtagctgggttggttatctgtctggtcgtctgcattcaacgcttcgcgcaagcgcaacagcagctcccattggaaagtctcggctgggacgtcgcagagcttcagctcaaccatactggtccacagcaagacccacggctgtactggcaggggggtcctgcattggggcgcagtttcttgcatggacctgagctggataaaggtcaattgcgcatccaccgagatggcatatacatggtgcatatccaggtcacgctcgccatttgctcttcaacaacggcgagcagacatcatcctacgacgctggcggtggggatttgttctcccgctagtcggtcaataagtttgctgcgactctcttttcaccagggctgcacaatcgtcagtcagaggctgacgcctctggcaaggggcgataccctttgtacaaacttgaccgggacccttctcccgagtagaaataccgatgaaacttttttcggagtccaatgggtgcgcccaaaaaacccggatccgtgggcgaaaaacctgaacgaaaaagattattga

SEQ ID NO: 16 - protein, 208 aa, Homo sapiens
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIVSQRLTPLARGDTLCTNLTGTLLPSRNTDETFFGVQWVRPKNPDPWAKNLNEKDY

SEQ ID NO: 17 - DNA, 1455 bp, Homo sapiens
  source 1..1455: mol_type = genomic DNA
atggagactgatacattgttgctttgggtccttttgttgtgggttcctggttctacgggcgcggcatcacaggtccaacttgttgaaagtggcgggggtgtcgtacagcccggacggtccttgcgattggactgtaaggcatctgggattaccttctcaaattccggtatgcattgggttcgccaagccccgggcaagggtctcgaatgggtggctgtaatttggtatgacgggtccaagagatactacgctgatagtgtgaagggtagatttacaatatcacgcgataattcaaaaaatacactgtttctgcagatgaacagcctgagggcagaggacacagcagtgtattattgcgctaccaatgatgattattgggggcagggtactctggtcactgtttcaagtggtggtggtggttcaggtggtgggggatctggcgggggcggaagtgagattgttctcactcaatcacccgcaaccctgtccttgtcccccggagaacgcgcgaccctgtcatgccgcgctagtcaatctgtcagtagttacctcgcatggtatcaacagaagcccggacaagctccacggctcctcatctacgatgcctcaaacagggcgacaggaatacccgcacgcttctcagggtccggtagcggtactgactttactttgactatatcttccctcgagccggaagatttcgcagtttactattgccaacagtccagtaactggccaagaacgtttggacaaggaacaaaagttgagattaagcgcacccacacgtgccccccttgcccagcacccgaagccgcaggtggcccatcagtgtttctttttcctccaaaaccaaaagacacactcatgatctcccggacgcctgaggtgacctgtgtagtcgtagacgtatcccatgaggaccctgaagtaaagtttaactggtatgtagacggtgtggaagtacacaatgccaagactaaaccaagagaggaacagtataacagcacctatagggtagtttccgtgctcaccgttctccaccaagattggcttaacggtaaagaatataaatgtaaggtgtcaaataaggcactcggagccccgatcgaaaagaccatctctaaagcaaaaggacagcccagggagccacaagtctacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 18 - protein, 484 aa, Homo sapiens
METDTLLLWVLLLWVPGSTGAASQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 2800 aa, Homo sapiens
TAATACGACTCACTATAAGGAGAAAGCTTACATTTGCTTCTGACACAACTGTGTTCACTAGCAACCTCAAACAGACACCATGGAGACAGACACACTCCTGCTATGGGTACTGCTGCTCTGGGTTCCAGGTTCCACTGGCGCCGCTAGCGATATTCAGATGACACAGTCACCGAGCTCCTTGTCTGCAAGCGTGGGGGACAGGGTTACCATTACTTGCCGGGCATCTCAGGACGTTAACACCGCAGTTGCATGGTACCAGCAGAAGCCCGGTAAAGCACCGAAACTCTTGATCTACTCAGCAAGTTTCTTGGAGTCTGGCGTACCAAGTAGATTCAGCGGTTCCAGATCAGGTACTGATTTCACGCTTACAATTTCTAGCTTGCAACCCGAGGATTTCGCGACTTACTACTGCCAGCAACACTATACAACACCCCCTACTTTTGGGCAGGGGACTAAAGTCGAGATAAAAGGCGGCGGTGGATCTGGTGGAGGTGGAAGCGGCGGAGGTGGCTCAGAAGTACAACTTGTTGAGTCCGGTGGTGGACTGGTCCAACCTGGCGGTTCACTTAGGCTGAGTTGCGCTGCATCAGGTTTTAATATCAAGGACACTTACATACATTGGGTCCGGCAGGCTCCAGGAAAAGGACTGGAATGGGTCGCCCGGATTTATCCAACCAATGGATATACAAGGTATGCGGATTCAGTTAAAGGAAGATTTACTATTTCCGCCGATACGTCCAAAAATACCGCTTACCTCCAGATGAATAGTCTTAGAGCTGAGGACACCGCCGTCTATTATTGCTCAAGGTGGGGTGGAGATGGGTTTTACGCTATGGATGTATGGGGGCAGGGCACCCTCGTTACCGTTTCAAGCGGGGGAGGCGGGTCTGGGGGAGGCGGAAGTGGGGGAGGAGGAAGCGAAGTTCAGCTGCTCGAATCCGGCGGCGGCCTTGTTCAGCCAGGTGGTAGCTTGAGGCTCAGTTGTGCTGCATCTGGGTTTACATTCTCAACTTATGCGATGAACTGGGTGAGGCAAGCACCTGGAAAGGGACTTGAGTGGGTCTCAAGAATTCGCTCCAAATACAACAACTATGCGACGTATTACGCAGACTCAGTGAAAGGACGGTTTACGATATCACGGGACGATTCAAAGAATACACTGTATTTGCAGATGAATTCTCTTAGGGCCGAAGACACTGCCGTATACTATTGTGTACGCCACGGTAATTTTGGCAATAGCTATGTATCTTGGTTCGCGTACTGGGGCCAAGGCACCCTTGTTACTGTGTCTAGTGGGGGCGGGGGGAGTGGTGGCGGAGGAAGTGGCGGGGGGGGATCTCAAGCGGTGGTTACTCAAGAGCCCTCCCTTACTGTTTCTCCGGGCGGGACGGTCACCTTGACTTGTGGCAGTTCAACAGGGGCAGTCACTACTAGTAATTATGCGAATTGGGTCCAAGAAAAGCCGGGCCAAGCTTTCCGGGGACTCATCGGAGGAACAAATAAAAGGGCACCCGGCACACCCGCGCGCTTTTCCGGGAGTCTTCTGGGCGGCAAGGCAGCCCTCACTCTCTCTGGGGCTCAACCTGAGGACGAGGCTGAGTACTATTGTGCCCTCTGGTACTCAAACCTGTGGGTCTTTGGAGGGGGAACCAAGCTTACGGTCTTGTCTAGAGAAAACCTGTATTTTCAGGGCACCCACACGTGCCCCCCTTGCCCAGCACCCGAAGCCGCAGGTGGCCCATCAGTGTTTCTTTTTCCTCCAAAACCAAAAGACACACTCATGATCTCCCGGACGCCTGAGGTGACCTGTGTAGTCGTAGACGTATCCCATGAGGACCCTGAAGTAAAGTTTAACTGGTATGTAGACGGTGTGGAAGTACACAATGCCAAGACTAAACCAAGAGAGGAACAGTATAACAGCACCTATAGGGTAGTTTCCGTGCTCACCGTTCTCCACCAAGATTGGCTTAACGGTAAAGAATATAAATGTAAGGTGTCAAATAAGGCACTCGGAGCCCCGATCGAAAAGACCATCTCTAAAGCAAAAGGACAGCCCAGGGAGCCACAAGTCTACACCCTGCCCCCATCCCGGGATGAGCTGACCAAGAACCAGGTCAGCCTGACCTGCCTGGTCAAAGGCTTCTATCCCAGCGACATCGCCGTGGAGTGGGAGAGCAATGGGCAGCCGGAGAACAACTACAAGACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCTCCGTGATGCATGAGGCTCTGCACAACCACTACACGCAGAAGAGCCTCTCCCTGTCTCCCGGGAAATGAGCTCGCTTTCTTGCTGTCCAATTTCTATTAAAGGTTCCTTTGTTCCCTAAGTCCAACTACTAAACTGGGGGATATTATGAAGGGCCTTGAGCATCTGGATTCTGCCTAATAAAAAACATTTATTTTCATTGCAGCTCGCTTTCTTGCTGTCCAATTTCTATTAAAGGTTCCTTTGTTCCCTAAGTCCAACTACTAAACTGGGGGATATTATGAAGGGCCTTGAGCATCTGGATTCTGCCTAATAAAAAACATTTATTTTCATTGCAGTCGACTCTAGAAAAAAAAAAAAAAAAAAAAAAAAAAAAAAAAAAAAAAAAAGGATCCCCGGGCGAGCTCCCAAAAAAAAAAAAAAAAAAAAAAAAAAAAAACCGAATTCCTGCAGCTCGAGAAAAAAAAAAAAAAAAAAAAAAAAAAAAAAAAAAAAAAAAA

SEQ ID NO: 20 - protein, 763 aa, Homo sapiens
METDTLLLWVLLLWVPGSTGAASDIQMTQSPSSLSASVGDRVTITCRASQDVNTAVAWYQQKPGKAPKLLIYSASFLESGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSGGGGSGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSGGGGSGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSRENLYFQGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - DNA, 60 bp, Homo sapiens
  source 1..60: mol_type = genomic DNA
atggagacagacacactcctgctatgggtactgctgctctgggttccaggttccactggc

SEQ ID NO: 22 - protein, 20 aa, Homo sapiens
METDTLLLWVLLLWVPGSTG

SEQ ID NO: 23 - DNA, 726 bp, Homo sapiens
  source 1..726: mol_type = genomic DNA
gatattcagatgacacagtcaccgagctccttgtctgcaagcgtgggggacagggttaccattacttgccgggcatctcaggacgttaacaccgcagttgcatggtaccagcagaagcccggtaaagcaccgaaactcttgatctactcagcaagtttcttggagtctggcgtaccaagtagattcagcggttccagatcaggtactgatttcacgcttacaatttctagcttgcaacccgaggatttcgcgacttactactgccagcaacactatacaacaccccctacttttgggcaggggactaaagtcgagataaaaggcggcggtggatctggtggaggtggaagcggcggaggtggctcagaagtacaacttgttgagtccggtggtggactggtccaacctggcggttcacttaggctgagttgcgctgcatcaggttttaatatcaaggacacttacatacattgggtccggcaggctccaggaaaaggactggaatgggtcgcccggatttatccaaccaatggatatacaaggtatgcggattcagttaaaggaagatttactatttccgccgatacgtccaaaaataccgcttacctccagatgaatagtcttagagctgaggacaccgccgtctattattgctcaaggtggggtggagatgggttttacgctatggatgtatgggggcagggcaccctcgttaccgtttcaagc

SEQ ID NO: 24 - protein, 242 aa, Homo sapiens
DIQMTQSPSSLSASVGDRVTITCRASQDVNTAVAWYQQKPGKAPKLLIYSASFLESGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 25 - DNA, 321 bp, Homo sapiens
  source 1..321: mol_type = genomic DNA
gatattcagatgacacagtcaccgagctccttgtctgcaagcgtgggggacagggttaccattacttgccgggcatctcaggacgttaacaccgcagttgcatggtaccagcagaagcccggtaaagcaccgaaactcttgatctactcagcaagtttcttggagtctggcgtaccaagtagattcagcggttccagatcaggtactgatttcacgcttacaatttctagcttgcaacccgaggatttcgcgacttactactgccagcaacactatacaacaccccctacttttgggcaggggactaaagtcgagataaaa

SEQ ID NO: 26 - protein, 107 aa, Homo sapiens
DIQMTQSPSSLSASVGDRVTITCRASQDVNTAVAWYQQKPGKAPKLLIYSASFLESGVPSRFSGSRSGTDFTLTISSLQPEDFATYYCQQHYTTPPTFGQGTKVEIK

SEQ ID NO: 27 - DNA, 45 bp, synthetic construct
  source 1..45: mol_type = other DNA
ggcggcggtggatctggtggaggtggaagcggcggaggtggctca

SEQ ID NO: 28 - protein, 15 aa, synthetic construct
GGGGSGGGGSGGGGS

SEQ ID NO: 29 - DNA, 360 bp, Homo sapiens
  source 1..360: mol_type = genomic DNA
gaagtacaacttgttgagtccggtggtggactggtccaacctggcggttcacttaggctgagttgcgctgcatcaggttttaatatcaaggacacttacatacattgggtccggcaggctccaggaaaaggactggaatgggtcgcccggatttatccaaccaatggatatacaaggtatgcggattcagttaaaggaagatttactatttccgccgatacgtccaaaaataccgcttacctccagatgaatagtcttagagctgaggacaccgccgtctattattgctcaaggtggggtggagatgggttttacgctatggatgtatgggggcagggcaccctcgttaccgtttcaagc

SEQ ID NO: 30 - protein, 120 aa, Homo sapiens
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 31 - protein, 45 aa, synthetic construct
GGGGGAGGCGGGTCTGGGGGAGGCGGAAGTGGGGGAGGAGGAAGC

SEQ ID NO: 32 - DNA, 747 bp, Homo sapiens
  source 1..747: mol_type = genomic DNA
gaagttcagctgctcgaatccggcggcggccttgttcagccaggtggtagcttgaggctcagttgtgctgcatctgggtttacattctcaacttatgcgatgaactgggtgaggcaagcacctggaaagggacttgagtgggtctcaagaattcgctccaaatacaacaactatgcgacgtattacgcagactcagtgaaaggacggtttacgatatcacgggacgattcaaagaatacactgtatttgcagatgaattctcttagggccgaagacactgccgtatactattgtgtacgccacggtaattttggcaatagctatgtatcttggttcgcgtactggggccaaggcacccttgttactgtgtctagtgggggcggggggagtggtggcggaggaagtggcggggggggatctcaagcggtggttactcaagagccctcccttactgtttctccgggcgggacggtcaccttgacttgtggcagttcaacaggggcagtcactactagtaattatgcgaattgggtccaagaaaagccgggccaagctttccggggactcatcggaggaacaaataaaagggcacccggcacacccgcgcgcttttccgggagtcttctgggcggcaaggcagccctcactctctctggggctcaacctgaggacgaggctgagtactattgtgccctctggtactcaaacctgtgggtctttggagggggaaccaagcttacggtcttg

SEQ ID NO: 33 - protein, 249 aa, Homo sapiens
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSGGGGSGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 34 - DNA, 375 bp, Homo sapiens
  source 1..375: mol_type = genomic DNA
gaagttcagctgctcgaatccggcggcggccttgttcagccaggtggtagcttgaggctcagttgtgctgcatctgggtttacattctcaacttatgcgatgaactgggtgaggcaagcacctggaaagggacttgagtgggtctcaagaattcgctccaaatacaacaactatgcgacgtattacgcagactcagtgaaaggacggtttacgatatcacgggacgattcaaagaatacactgtatttgcagatgaattctcttagggccgaagacactgccgtatactattgtgtacgccacggtaattttggcaatagctatgtatcttggttcgcgtactggggccaaggcacccttgttactgtgtctagt

SEQ ID NO: 35 - protein, 125 aa, Homo sapiens
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 36 - DNA, 45 bp, synthetic construct
  source 1..45: mol_type = other DNA
gggggcggggggagtggtggcggaggaagtggcggggggggatct

SEQ ID NO: 37 - DNA, 327 bp, Homo sapiens
  source 1..327: mol_type = genomic DNA
caagcggtggttactcaagagccctcccttactgtttctccgggcgggacggtcaccttgacttgtggcagttcaacaggggcagtcactactagtaattatgcgaattgggtccaagaaaagccgggccaagctttccggggactcatcggaggaacaaataaaagggcacccggcacacccgcgcgcttttccgggagtcttctgggcggcaaggcagccctcactctctctggggctcaacctgaggacgaggctgagtactattgtgccctctggtactcaaacctgtgggtctttggagggggaaccaagcttacggtcttg

SEQ ID NO: 38 - protein, 109 aa, Homo sapiens
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 39 - DNA, 705 bp, Homo sapiens
  source 1..705: mol_type = genomic DNA
tctagagaaaacctgtattttcagggcacccacacgtgccccccttgcccagcacccgaagccgcaggtggcccatcagtgtttctttttcctccaaaaccaaaagacacactcatgatctcccggacgcctgaggtgacctgtgtagtcgtagacgtatcccatgaggaccctgaagtaaagtttaactggtatgtagacggtgtggaagtacacaatgccaagactaaaccaagagaggaacagtataacagcacctatagggtagtttccgtgctcaccgttctccaccaagattggcttaacggtaaagaatataaatgtaaggtgtcaaataaggcactcggagccccgatcgaaaagaccatctctaaagcaaaaggacagcccagggagccacaagtctacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 40 - protein, 234 aa, Homo sapiens
SRENLYFQGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - DNA, 2785 bp, Homo sapiens
  source 1..2785: mol_type = genomic DNA
taatacgactcactataaggagaaagcttacatttgcttctgacacaactgtgttcactagcaacctcaaacagacaccatggagacagacacactcctgctatgggtactgctgctctgggttccaggttccactggcgccgctagccaggtgcaactcgtacaaagcggttccgaactgaagaaacctggtgcgagcgtaaaggtgtcctgtaaagcatctggatacaccttcaccaactacggaatgaactgggttcgacaggcaccgggccaaggcttggagtggatggggtggatcaacacctacacgggtgaacctacgtacgcagatgatttcaaaggtcgcttcgtgttttccctggatacttctgtgtccacagcgtacctccagatttccagtctgaaggccgaggacacagccgtatattactgtgctcggtggttgcgcgattttgattattggggcgcaggtactactgtgacagtaagctccggcggaggcgggtcagggggcggtggatccggcggagggggatcagaaatcgtgctcactcaatcaccagcgaccttgagcttgtcacctggggagcgggcgacgctgtcctgtagtgcgtcatcatctatttcatatatgcactggtatcaacagaagccaggacaggcgcctcgcctgttgatttacgatactagcaaattggcaacggggatacccgctcgattcagcggcagcggatctgggacggatttcacattgaccatttctagccttgaaccagaggacttcgcagtatattactgccatcagaggtccagctatccatacacatttggcggagggacgaaattggaaatcaaggggggaggcgggtctgggggaggcggaagtgggggaggaggaagcgaagttcagctgctcgaatccggcggcggccttgttcagccaggtggtagcttgaggctcagttgtgctgcatctgggtttacattctcaacttatgcgatgaactgggtgaggcaagcacctggaaagggacttgagtgggtctcaagaattcgctccaaatacaacaactatgcgacgtattacgcagactcagtgaaaggacggtttacgatatcacgggacgattcaaagaatacactgtatttgcagatgaattctcttagggccgaagacactgccgtatactattgtgtacgccacggtaattttggcaatagctatgtatcttggttcgcgtactggggccaaggcacccttgttactgtgtctagtgggggcggggggagtggtggcggaggaagtggcggggggggatctcaagcggtggttactcaagagccctcccttactgtttctccgggcgggacggtcaccttgacttgtggcagttcaacaggggcagtcactactagtaattatgcgaattgggtccaagaaaagccgggccaagctttccggggactcatcggaggaacaaataaaagggcacccggcacacccgcgcgcttttccgggagtcttctgggcggcaaggcagccctcactctctctggggctcaacctgaggacgaggctgagtactattgtgccctctggtactcaaacctgtgggtctttggagggggaaccaagcttacggtcttgtctagagaaaacctgtattttcagggcacccacacgtgccccccttgcccagcacccgaagccgcaggtggcccatcagtgtttctttttcctccaaaaccaaaagacacactcatgatctcccggacgcctgaggtgacctgtgtagtcgtagacgtatcccatgaggaccctgaagtaaagtttaactggtatgtagacggtgtggaagtacacaatgccaagactaaaccaagagaggaacagtataacagcacctatagggtagtttccgtgctcaccgttctccaccaagattggcttaacggtaaagaatataaatgtaaggtgtcaaataaggcactcggagccccgatcgaaaagaccatctctaaagcaaaaggacagcccagggagccacaagtctacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaatgagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcagtcgactctagaaaaaaaaaaaaaaaaaaaaaaaaaaaaaaaaaaaaaaaaaggatccccgggcgagctcccaaaaaaaaaaaaaaaaaaaaaaaaaaaaaaccgaattcctgcagctcgagaaaaaaaaaaaaaaaaaaaaaaaaaaaaaaaaaaaaaaaaa

SEQ ID NO: 42 - protein, 758 aa, Homo sapiens
METDTLLLWVLLLWVPGSTGAASQVQLVQSGSELKKPGASVKVSCKASGYTFTNYGMNWVRQAPGQGLEWMGWINTYTGEPTYADDFKGRFVFSLDTSVSTAYLQISSLKAEDTAVYYCARWLRDFDYWGAGTTVTVSSGGGGSGGGGSGGGGSEIVLTQSPATLSLSPGERATLSCSASSSISYMHWYQQKPGQAPRLLIYDTSKLATGIPARFSGSGSGTDFTLTISSLEPEDFAVYYCHQRSSYPYTFGGGTKLEIKGGGGSGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSGGGGSGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSRENLYFQGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 116 aa, Homo sapiens
QVQLVQSGSELKKPGASVKVSCKASGYTFTNYGMNWVRQAPGQGLEWMGWINTYTGEPTYADDFKGRFVFSLDTSVSTAYLQISSLKAEDTAVYYCARWLRDFDYWGAGTTVTVSS

SEQ ID NO: 44 - protein, 106 aa, Homo sapiens
EIVLTQSPATLSLSPGERATLSCSASSSISYMHWYQQKPGQAPRLLIYDTSKLATGIPARFSGSGSGTDFTLTISSLEPEDFAVYYCHQRSSYPYTFGGGTKLEIK

SEQ ID NO: 45 - DNA, 2851 bp, Homo sapiens
  source 1..2851: mol_type = genomic DNA
taatacgactcactataaggagaaagcttacatttgcttctgacacaactgtgttcactagcaacctcaaacagacaccatggagacagacacactcctgctatgggtactgctgctctgggttccaggttccactggcgccgctagccaggtacagctgcagcagtcaggtccaggactcgtgacgccctcgcagaccctctcactcacctgtgccatctccggggacagtgtctctagcaacagtgctacttggaactggatcaggcagtccccatcgagaggccttgagtggctgggaaggacatactacaggtccaagtggtataacgactatgcagtatctgtgaaaagtcgaatgagcatcaacccagacacatccaagaaccagttctccctgcagctgaactctgtgactcccgaggacacggctgtgtattactgtgcaagaggaatgatgacttactattacggtatggacgtctggggccaagggaccacggtcaccgtctcctcaggcattctaggatccggtggcggtggcagcggcggtggtggttccggaggcggcggttctcagcctgtgctgactcagtcgtcttccctctctgcatctcctggagcatcagccagtctcacctgcaccttgcgcagtggcatcaatgttggtccctacaggatatactggtaccagcagaagccagggagtcctccccagtatctcctgaactacaaatcagactcagataagcagcagggctctggagtccccagccgcttctctggatccaaagatgcttcggccaatgcaggggttttactcatctctgggctccggtctgaggatgaggctgactattactgtatgatttggcacagcagcgctgctgtgttcggaggaggcacccaactgaccgtcctctccgggggaggcgggtctgggggaggcggaagtgggggaggaggaagcgaagttcagctgctcgaatccggcggcggccttgttcagccaggtggtagcttgaggctcagttgtgctgcatctgggtttacattctcaacttatgcgatgaactgggtgaggcaagcacctggaaagggacttgagtgggtctcaagaattcgctccaaatacaacaactatgcgacgtattacgcagactcagtgaaaggacggtttacgatatcacgggacgattcaaagaatacactgtatttgcagatgaattctcttagggccgaagacactgccgtatactattgtgtacgccacggtaattttggcaatagctatgtatcttggttcgcgtactggggccaaggcacccttgttactgtgtctagtgggggcggggggagtggtggcggaggaagtggcggggggggatctcaagcggtggttactcaagagccctcccttactgtttctccgggcgggacggtcaccttgacttgtggcagttcaacaggggcagtcactactagtaattatgcgaattgggtccaagaaaagccgggccaagctttccggggactcatcggaggaacaaataaaagggcacccggcacacccgcgcgcttttccgggagtcttctgggcggcaaggcagccctcactctctctggggctcaacctgaggacgaggctgagtactattgtgccctctggtactcaaacctgtgggtctttggagggggaaccaagcttacggtcttgtctagagaaaacctgtattttcagggcacccacacgtgccccccttgcccagcacccgaagccgcaggtggcccatcagtgtttctttttcctccaaaaccaaaagacacactcatgatctcccggacgcctgaggtgacctgtgtagtcgtagacgtatcccatgaggaccctgaagtaaagtttaactggtatgtagacggtgtggaagtacacaatgccaagactaaaccaagagaggaacagtataacagcacctatagggtagtttccgtgctcaccgttctccaccaagattggcttaacggtaaagaatataaatgtaaggtgtcaaataaggcactcggagccccgatcgaaaagaccatctctaaagcaaaaggacagcccagggagccacaagtctacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaatgagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcagtcgactctagaaaaaaaaaaaaaaaaaaaaaaaaaaaaaaaaaaaaaaaaaggatccccgggcgagctcccaaaaaaaaaaaaaaaaaaaaaaaaaaaaaaccgaattcctgcagctcgagaaaaaaaaaaaaaaaaaaaaaaaaaaaaaaaaaaaaaaaaa

SEQ ID NO: 46 - protein, 780 aa, Homo sapiens
METDTLLLWVLLLWVPGSTGAASQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSGGGGSGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSGGGGSGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSRENLYFQGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 123 aa, Homo sapiens
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSS

SEQ ID NO: 48 - protein, 115 aa, Homo sapiens
QPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVL